(19) United States
(12) Patent Application Publication
(10) Pub. No.: US 2025/0382587 A1
(43) Pub. Date: Dec. 18, 2025

(54) METHODS FOR MANUFACTURING VIRUSES AND VIRAL PARTICLES

(71) Applicant: Avirmax Biopharma Inc., Hayward, CA (US)

(72) Inventors: Shengjiang LIU, Lafayette, CA (US); Ningguang LUO, Castro Valley, CA (US)

(21) Appl. No.: 19/239,726

(22) Filed: Jun. 16, 2025

Related U.S. Application Data
(63) Continuation of application No. PCT/US2023/084355, filed on Dec. 15, 2023.
(60) Provisional application No. 63/433,338, filed on Dec. 16, 2022.

(51) Int. Cl. C12N 7/00; C12N 5/07; C12N 15/85

(52) U.S. Cl. CPC C12N 7/00; C12N 5/0601; C12N 15/85; C12N 2750/14143; C12N 2750/14152

(57) ABSTRACT

Described herein are methods for manufacturing viruses and viral particles. In some aspects, the methods use a sugar-based detergent for lysing a host cell to release the virus or viral particle. Also, described herein is a composition or a pharmaceutical composition comprising the virus or viral particle manufactured by the methods described herein.

### CROSS-REFERENCE

This application is a continuation of International Application No. PCT/US2023/084355, filed Dec. 15, 2023, which claims the benefit of U.S. Provisional Application Ser. No. 63/433,338 filed on Dec. 16, 2022, the entirety of which is hereby incorporated by reference herein.

### SEQUENCE LISTING

The instant application contains a Sequence Listing which has been submitted electronically in XML file format and is hereby incorporated by reference in its entirety. Said XML copy, created on Jun. 16, 2025, is named 59561-706.301_SL.xml and is 107,781 bytes in size.

### BACKGROUND

The use of virus for therapeutics has been one of the cornerstones of modern biomedicine. To manufacture sufficient quantity of virus for pharmaceutical use often requires propagating the virus in host cells. Host cells can be mammalians cells or insect cells. The host cells can be lysed by the use of detergent for releasing the virus. However, manufacturing of virus for therapeutic purposes in sufficiently large scale remains an ongoing challenge. For example, viral manufacturing processes rely on utilization of detergent that is harmful to the environment. Additionally, industrial scale of manufacturing virus or viral particles can lead to e.g., aggregation of the viral particles or harmful byproducts such as other viruses that are distinct from the desired viruses or viral particles.

### SUMMARY

There remains an unmet need for improved compositions, systems, and methods for manufacturing viruses and/or viral particles. This disclosure meets this unmet need.

Described herein, in some aspects, is a method of releasing a plurality of viruses from a host cell, the plurality of viruses are encoded by a vector in the host cell, the method comprising: contacting the host cell with a formulation comprising a sugar-based detergent for lysing the host cell, wherein said contacting with a sugar-based detergent releases at least 50% of the plurality of viruses from the host cell. In some embodiments, the plurality of viruses comprises non-enveloped viruses. In some embodiments, the non-enveloped viruses comprise DNA viruses. In some embodiments, the non-enveloped viruses comprise RNA viruses. In some embodiments, the vector encodes at least one virus comprising at least one capsid. In some embodiments, the at least one capsid comprises a capsid of serotype AAV1, AAV2, AAV3, AAV4, AAV5, AAV6, AAV7, AAV8, AAV9, AAV10, AAV11, AAV12, AAV13, or a combination thereof. In some embodiments, the vector encodes at least one therapeutic. In some embodiments, the contacting of the host cell with the formulation inactivates viruses that are different from the plurality of viruses. In some embodiments, the viruses being inactivated are not encoded by the vector. In some embodiments, the sugar-based detergent comprises at least one sugar moiety and at least one hydrophobic moiety. In some embodiments, the at least one sugar moiety comprises a sugar monomer. In some embodiments, the at least one sugar moiety comprises a sugar polymer. In some embodiments, the sugar polymer comprises at least two, at least three, at least four, at least five, at least six, at least seven, at least eight, at least nine, at least 10, at least 11, at least 12, or more sugar molecules. In some embodiments, the at least one sugar moiety comprises at least one glycoside. In some embodiments, the at least one glycoside is covalently connected to the at least one hydrophobic moiety. In some embodiments, the at least one hydrophobic moiety comprises a fatty acid tail. In some embodiments, the fatty acid tail comprises at least two, at least three, at least four, at least five, at least six, at least seven, at least eight, at least nine, at least 10, at least 11, at least 12, or more carbons as part of carbon chain. In some embodiments, the contacting with the sugar-based detergent releases at least 60%, at least 70%, at least 80%, at least 90%, or more of the plurality of viruses from the host cell. In some embodiments, the host cell is a bacteria cell. In some embodiments, the host cell is an eukaryotic cell. In some embodiments, the eukaryotic cell is a mammalian cell. In some embodiments, the eukaryotic cell is a yeast cell. In some embodiments, the eukaryotic cell is an insect cell. In some embodiments, the formulation comprises at least one additional active ingredient. In some embodiments, the at least one additional active ingredient comprises a non-ionic surfactant. In some embodiments, the at least one additional active ingredient comprises an anionic surfactant. In some embodiments, the method further comprises at least one of following for isolating the plurality of viruses from the host cell: filtration, chromatography, centrifugation, buffer exchange, or sterilization.

Described herein, in some aspects, is a method of releasing a plurality of viruses from a host cell, the plurality of viruses are encoded by a vector in the host cell, the method comprising: contacting the host cell with a sugar-based detergent for lysing the host cell, wherein said contacting with the a sugar-based detergent releases the plurality of viruses that are at least 50% more infective compared to an infectivity of a second plurality of comparable viruses that are obtained by releasing the second plurality of the comparable viruses by a method that does not use the sugar-based detergent. In some embodiments, the plurality of viruses, upon released by contacting with the sugar-based detergent, is at least 60%, at least 70%, at least 80%, at least 90%, or more infective compared to the infectivity of the second plurality of comparable viruses. In some embodiments, the infectivity is determined by a viral titer assay. In some embodiments, the second plurality of comparable viruses, after being released from a comparable hose cell, forms at least 10% more aggregate compared by the plurality of viruses.

Described herein, in some aspects, is a method of releasing a plurality of viruses from a host cell cultured in a medium, where the plurality of viruses are encoded by a vector in the host cell, the method comprising contacting of the host cell with a sugar-based detergent to the medium so that the medium comprises at least 0.1% of the sugar-based detergent, where the medium comprising the at least 0.1% of the sugar-based detergent lyses the host cell and releases the plurality of viruses from the host cell.

Described herein, in some aspects, is a method of releasing a plurality of viruses from a host cell, the plurality of viruses are encoded by a vector in the host cell, the method comprising: contacting the host cell with a formulation comprising a sugar-based detergent for lysing the host cell, wherein said contacting with a biodegradable detergent release at least 50% of the plurality of viruses from the host cell.

Described herein, in some aspects, is a pharmaceutical composition comprising the plurality of viruses described herein. In some embodiments, the pharmaceutical composition is in unit dose form. In some embodiments, the pharmaceutical composition further comprises a pharmaceutical acceptable excipient. In some embodiments, the pharmaceutical composition further comprises at least one active ingredient. In some embodiments, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof.

Described herein, in some aspects, is a kit comprising a plurality of viruses described herein or the pharmaceutical composition described herein; and a container.

Described herein, in some aspects, is a method for treating a disease or condition in a subject in need thereof, the method comprising administering a therapeutically effective amount of the plurality of viruses described herein or the pharmaceutical composition described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 illustrates an exemplary process for manufacturing of a viral particle (e.g., a virus released from a host cell by contacting the host cell with a sugar-based detergent described herein).

FIG. 2 illustrates examples of wells counted as positive (A, B, and C) and negative (D) in the F-TCID50 assay.

FIG. 3 illustrates optimization of cell density at the time of inoculation during the rBV F-TCID50 assay.

FIG. 4 illustrates baculovirus inactivation by AAVx column eluate pH=3.0.

FIG. 5 illustrates SDS-PAGE and SimplyBlue staining of the purified AAV vectors.

FIG. 6 illustrates recovery of AAV by contacting the Sf9 host cells with a sugar-based detergent (e.g., an APG described herein) supplemented with NaCl compared with Triton X-100 supplemented with NaCl.

FIG. 7 illustrates Sf9 cell growth curve (left) and AAV production (right).

FIG. 8 illustrates potency of VEGF-Trap expressed by rAAV vectors (AVMX-110) purified from Sf9 cell culture lysed with APG.

FIG. 9 illustrates turbidity assay of an exemplary scale-up manufacturing process.

FIG. 10 illustrates an exemplary schematic and turbidity graph for dissociation of AAV (AVMX-102) from Sf9 cell lysate.

FIG. 11 illustrates yield as determined by protein concentration (left) and qPCR targeting AAV (right).

FIG. 12 illustrates Sf9 cell lysates optical density before and after microfluidics treatment.

FIG. 13 illustrates Sf9 cells before microfluidics (left) or Sf9 cell debris after microfluidics (right).

### DETAILED DESCRIPTION

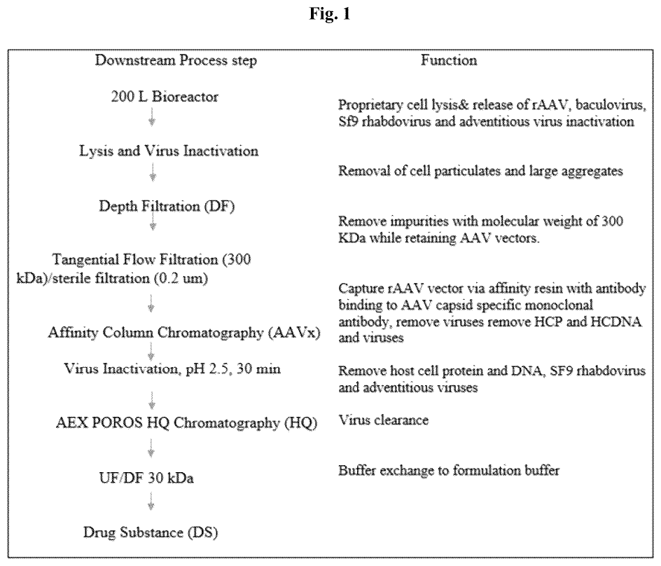

Described herein, in some aspects, are methods for viral manufacturing. In some embodiments, the methods comprise releasing a plurality of viruses or viral particles from a host cell. In some embodiments, the plurality of viruses or viral particles are released from the host cell by contacting the host cell with a sugar-based detergent or a formulation comprising the sugar-based detergent. In some embodiments, the sugar-based detergent is a biodegradable detergent, where the biodegradable detergent is safe for the environment (e.g., safe per the environmental standard set by regulatory agencies). In some embodiments, the contacting with the sugar-based detergent can release the plurality of viruses or viral particles directly from the host cell. For example, the contacting of the host cell with the sugar-based detergent can occur in a liquid suspension (e.g., a cell culture environment for the host cell) without the need to concentrate or pellet the host cell. Such arrangement presents an improvement over the methods currently used for viral manufacturing, where pelleting and mechanical pressure used for releasing the viral particles can lead to aggregation of the viral particles and decreased yields. In some embodiments, the contacting of the host cell with the sugar-based detergent can simultaneously inactivate other viruses (e.g., viruses not encoded by a vector described herein). In some embodiments, the methods described herein include other purification modalities such as filtration, chromatography, centrifugation, buffer exchange, or sterilization. FIG. 1 illustrates an exemplary process for manufacturing of a viral particle (e.g., a virus released from a host cell by contacting the host cell with a sugar-based detergent described herein).

In some embodiments, the plurality of viruses or viral particles released from the host cell is encoded by a vector in the host cell. For example, the plurality of viruses being manufactured and released from the host cell are viruses encoded from a vector comprising an engineered polynucleotide transduced into the host cell. In some embodiments, the plurality of viruses or viral particles comprises an engineered capsid described herein. In some embodiments, the plurality of viruses or viral particles comprises non-enveloped viruses. In some embodiments, the plurality of viruses or viral particles can be used to treat a disease or condition in a subject. For example, the plurality of viruses or viral particles can be a vehicle for delivering gene therapy or a therapeutic to a cell in a subject for treating the disease or condition. In some embodiments, the plurality of viruses or viral particles can be formulated into a pharmaceutical composition or a pharmaceutical formulation. In some embodiments, the plurality of viruses or viral particles can modify a cell in the subject. For example, the plurality of viruses or viral particles can deliver a gene editing construct into the cell of the subject.

In some embodiments, the vector comprises an engineered polynucleotide described herein. In some embodiments, the vector is an engineered polynucleotide described herein. In some embodiments, the engineered polynucleotide described herein can be codon optimized. In some embodiments, the engineered polynucleotide comprises two or more expression cassettes for expressing two or more peptides, fusion proteins, therapeutics, or a combination thereof. In some embodiments, the engineered polynucleotide encodes an engineered polypeptide (e.g., an engineered capsid) described herein. In some embodiments, the engineered polynucleotide encodes an engineered capsid described herein.

### Sugar-Based Detergent

Described herein, in some aspects, are methods for manufacturing the plurality of viruses or viral particles described herein from a host cell. In some embodiments, the method comprises contacting a host cell with a sugar-based detergent described herein to release the plurality of viruses or viral particles from the host cell. In some embodiments, the sugar-based detergent is biodegradable. In some embodiments, the sugar-based detergent is environmentally safe. Non-limiting examples of sugar-based detergents can include sorbitan esters, sucrose esters, alkyl polyglycosides (APG), or fatty acid glucamides. In some embodiments, the sugar-based detergent is an APG. In some embodiments, the sugar-based detergent is a derivative of APG.

In some embodiments, the sugar-based detergent comprises at least one sugar moiety. In some embodiments, the sugar-based detergent comprises at least one hydrophobic moiety. In some embodiments, the sugar-based detergent comprises at least one sugar moiety and at least one hydrophobic moiety. In some embodiments, the at least one sugar moiety comprises a sugar monomer. In some embodiments, the at least one sugar moiety comprises a sugar polymer. In some embodiments, the sugar polymer comprises at least two, at least three, at least four, at least five, at least six, at least seven, at least eight, at least nine, at least 10, at least 11, at least 12, or more sugar molecules. In some embodiments, the sugar polymer comprises between about 2 sugar molecules to about 15 sugar molecules. In some embodiments, the sugar polymer comprises between about 2 sugar molecules to about 3 sugar molecules, about 2 sugar molecules to about 4 sugar molecules, about 2 sugar molecules to about 5 sugar molecules, about 2 sugar molecules to about 6 sugar molecules, about 2 sugar molecules to about 7 sugar molecules, about 2 sugar molecules to about 8 sugar molecules, about 2 sugar molecules to about 9 sugar molecules, about 2 sugar molecules to about 10 sugar molecules, about 2 sugar molecules to about 11 sugar molecules, about 2 sugar molecules to about 12 sugar molecules, about 2 sugar molecules to about 15 sugar molecules, about 3 sugar molecules to about 4 sugar molecules, about 3 sugar molecules to about 5 sugar molecules, about 3 sugar molecules to about 6 sugar molecules, about 3 sugar molecules to about 7 sugar molecules, about 3 sugar molecules to about 8 sugar molecules, about 3 sugar molecules to about 9 sugar molecules, about 3 sugar molecules to about 10 sugar molecules, about 3 sugar molecules to about 11 sugar molecules, about 3 sugar molecules to about 12 sugar molecules, about 3 sugar molecules to about 15 sugar molecules, about 4 sugar molecules to about 5 sugar molecules, about 4 sugar molecules to about 6 sugar molecules, about 4 sugar molecules to about 7 sugar molecules, about 4 sugar molecules to about 8 sugar molecules, about 4 sugar molecules to about 9 sugar molecules, about 4 sugar molecules to about 10 sugar molecules, about 4 sugar molecules to about 11 sugar molecules, about 4 sugar molecules to about 12 sugar molecules, about 4 sugar molecules to about 15 sugar molecules, about 5 sugar molecules to about 6 sugar molecules, about 5 sugar molecules to about 7 sugar molecules, about 5 sugar molecules to about 8 sugar molecules, about 5 sugar molecules to about 9 sugar molecules, about 5 sugar molecules to about 10 sugar molecules, about 5 sugar molecules to about 11 sugar molecules, about 5 sugar molecules to about 12 sugar molecules, about 5 sugar molecules to about 15 sugar molecules, about 6 sugar molecules to about 7 sugar molecules, about 6 sugar molecules to about 8 sugar molecules, about 6 sugar molecules to about 9 sugar molecules, about 6 sugar molecules to about 10 sugar molecules, about 6 sugar molecules to about 11 sugar molecules, about 6 sugar molecules to about 12 sugar molecules, about 6 sugar molecules to about 15 sugar molecules, about 7 sugar molecules to about 8 sugar molecules, about 7 sugar molecules to about 9 sugar molecules, about 7 sugar molecules to about 10 sugar molecules, about 7 sugar molecules to about 11 sugar molecules, about 7 sugar molecules to about 12 sugar molecules, about 7 sugar molecules to about 15 sugar molecules, about 8 sugar molecules to about 9 sugar molecules, about 8 sugar molecules to about 10 sugar molecules, about 8 sugar molecules to about 11 sugar molecules, about 8 sugar molecules to about 12 sugar molecules, about 8 sugar molecules to about 15 sugar molecules, about 9 sugar molecules to about 10 sugar molecules, about 9 sugar molecules to about 11 sugar molecules, about 9 sugar molecules to about 12 sugar molecules, about 9 sugar molecules to about 15 sugar molecules, about 10 sugar molecules to about 11 sugar molecules, about 10 sugar molecules to about 12 sugar molecules, about 10 sugar molecules to about 15 sugar molecules, about 11 sugar molecules to about 12 sugar molecules, about 11 sugar molecules to about 15 sugar molecules, or about 12 sugar molecules to about 15 sugar molecules. In some embodiments, the sugar polymer comprises between about 2 sugar molecules, about 3 sugar molecules, about 4 sugar molecules, about 5 sugar molecules, about 6 sugar molecules, about 7 sugar molecules, about 8 sugar molecules, about 9 sugar molecules, about 10 sugar molecules, about 11 sugar molecules, about 12 sugar molecules, or about 15 sugar molecules. In some embodiments, the sugar polymer comprises between at least about 2 sugar molecules, about 3 sugar molecules, about 4 sugar molecules, about 5 sugar molecules, about 6 sugar molecules, about 7 sugar molecules, about 8 sugar molecules, about 9 sugar molecules, about 10 sugar molecules, about 11 sugar molecules, or about 12 sugar molecules. In some embodiments, the sugar polymer comprises between at most about 3 sugar molecules, about 4 sugar molecules, about 5 sugar molecules, about 6 sugar molecules, about 7 sugar molecules, about 8 sugar molecules, about 9 sugar molecules, about 10 sugar molecules, about 11 sugar molecules, about 12 sugar molecules, or about 15 sugar molecules.

In some embodiments, the at least one sugar moiety comprises at least one glycoside. In some embodiments, the at least one sugar moiety comprises at least two glycosides. In some embodiments, the at least one sugar moiety comprises at least three glycosides. In some embodiments, the at least one sugar moiety comprises at least four glycosides. In some embodiments, the at least one sugar moiety comprises at least five glycosides. In some embodiments, the at least one sugar moiety comprises at least six glycosides. In some embodiments, the at least one glycoside is covalently connected to the at least one hydrophobic moiety. In some embodiments, the at least one hydrophobic moiety comprises a fatty acid tail. In some embodiments, the fatty acid tail comprises at least two, at least three, at least four, at least five, at least six, at least seven, at least eight, at least nine, at least 10, at least 11, at least 12, or more carbons as part of carbon chain. In some embodiments, the fatty acid tail comprises between about 2 carbons to about 15 carbons. In some embodiments, the fatty acid tail comprises between about 2 carbons to about 3 carbons, about 2 carbons to about 4 carbons, about 2 carbons to about 5 carbons, about 2 carbons to about 6 carbons, about 2 carbons to about 7 carbons, about 2 carbons to about 8 carbons, about 2 carbons to about 9 carbons, about 2 carbons to about 10 carbons, about 2 carbons to about 11 carbons, about 2 carbons to about 12 carbons, about 2 carbons to about 15 carbons, about 3 carbons to about 4 carbons, about 3 carbons to about 5 carbons, about 3 carbons to about 6 carbons, about 3 carbons to about 7 carbons, about 3 carbons to about 8 carbons, about 3 carbons to about 9 carbons, about 3 carbons to about 10 carbons, about 3 carbons to about 11 carbons, about 3 carbons to about 12 carbons, about 3 carbons to about 15 carbons, about 4 carbons to about 5 carbons, about 4 carbons to about 6 carbons, about 4 carbons to about 7 carbons, about 4 carbons to about 8 carbons, about 4 carbons to about 9 carbons, about 4 carbons to about 10 carbons, about 4 carbons to about 11 carbons, about 4 carbons to about 12 carbons, about 4 carbons to about 15 carbons, about 5 carbons to about 6 carbons, about 5 carbons to about 7 carbons, about 5 carbons to about 8 carbons, about 5 carbons to about 9 carbons, about 5 carbons to about 10 carbons, about 5 carbons to about 11 carbons, about 5 carbons to about 12 carbons, about 5 carbons to about 15 carbons, about 6 carbons to about 7 carbons, about 6 carbons to about 8 carbons, about 6 carbons to about 9 carbons, about 6 carbons to about 10 carbons, about 6 carbons to about 11 carbons, about 6 carbons to about 12 carbons, about 6 carbons to about 15 carbons, about 7 carbons to about 8 carbons, about 7 carbons to about 9 carbons, about 7 carbons to about 10 carbons, about 7 carbons to about 11 carbons, about 7 carbons to about 12 carbons, about 7 carbons to about 15 carbons, about 8 carbons to about 9 carbons, about 8 carbons to about 10 carbons, about 8 carbons to about 11 carbons, about 8 carbons to about 12 carbons, about 8 carbons to about 15 carbons, about 9 carbons to about 10 carbons, about 9 carbons to about 11 carbons, about 9 carbons to about 12 carbons, about 9 carbons to about 15 carbons, about 10 carbons to about 11 carbons, about 10 carbons to about 12 carbons, about 10 carbons to about 15 carbons, about 11 carbons to about 12 carbons, about 11 carbons to about 15 carbons, or about 12 carbons to about 15 carbons. In some embodiments, the fatty acid tail comprises between about 2 carbons, about 3 carbons, about 4 carbons, about 5 carbons, about 6 carbons, about 7 carbons, about 8 carbons, about 9 carbons, about 10 carbons, about 11 carbons, about 12 carbons, or about 15 carbons. In some embodiments, the fatty acid tail comprises between at least about 2 carbons, about 3 carbons, about 4 carbons, about 5 carbons, about 6 carbons, about 7 carbons, about 8 carbons, about 9 carbons, about 10 carbons, about 11 carbons, or about 12 carbons. In some embodiments, the fatty acid tail comprises between at most about 3 carbons, about 4 carbons, about 5 carbons, about 6 carbons, about 7 carbons, about 8 carbons, about 9 carbons, about 10 carbons, about 11 carbons, about 12 carbons, or about 15 carbons.

In some embodiments, the sugar-based detergent described herein comprises a structure of:

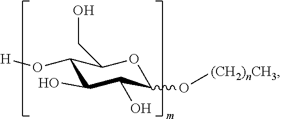

where m and n are each an integer. In some embodiments, m comprises an integer of 0, 1, 2, 3, 4, 5, 6, 7, 8, 9, 10, 11, 12, 13, 14, 15, 16, 17, 18, 19, or 20. In some embodiments, n comprises an integer of 0, 1, 2, 3, 4, 5, 6, 7, 8, 9, 10, 11, 12, 13, 14, 15, 16, 17, 18, 19, or 20. In some embodiments, m and n each comprises an integer of 0, 1, 2, 3, 4, 5, 6, 7, 8, 9, 10, 11, 12, 13, 14, 15, 16, 17, 18, 19, or 20. In some embodiments, m is an integer of 4. In some embodiments, m is an integer of 5. In some embodiments, m is an integer of 6. In some embodiments, m is an integer of 7. In some embodiments, m is an integer of 8. In some embodiments, m is an integer of 9. In some embodiments, m is an integer of 10. In some embodiments, m is an integer of 11. In some embodiments, m is an integer of 12. In some embodiments, n is an integer of 4. In some embodiments, n is an integer of 5. In some embodiments, n is an integer of 6. In some embodiments, n is an integer of 7. In some embodiments, n is an integer of 8. In some embodiments, n is an integer of 9. In some embodiments, n is an integer of 10. In some embodiments, n is an integer of 11. In some embodiments, n is an integer of 12. In some embodiments, m is an integer of 8, and n is an integer of 9.

In some embodiments, the contacting of the host cell with the sugar-based detergent releases at least 10%, at least 20%, at least 30%, at least 40%, at least 50%, at least 60%, at least 70%, at least 80%, at least 90%, or more of the plurality of viruses or viral particles encoded by a vector described herein from the host cell. In some embodiments, the contacting of the host cell with the sugar-based detergent increases the yield of the plurality of viruses or viral particles released from the host cell compared to other methods not using the sugar-based detergent. For example, the contacting of the host cell with the sugar-based detergent increases yield of the plurality of viruses or viral particles released from the host cell compared to yield of releasing the same plurality of viruses or viral particles from a comparable host cell by contacting the comparable host cell with a non-sugar-based detergent (e.g., Triton X-100). In some embodiments, the contacting of the host cell with the sugar-based detergent increases the yield of the plurality of viruses or viral particles released from the host cell by at least 0.1 fold, at least 0.2 fold, at least 0.4 fold, at least 0.8 fold, at least 1.0 fold, at least 1.2 fold, at least 1.5 fold, at least 2.0 fold, at least 3.0 fold, at least 4.0 fold, at least 5.0 fold, at least 10.0 fold, at least 20.0 fold, at least 50.0 fold, at least 100.0 fold, or more fold compared to yield of releasing the same plurality of viruses or viral particles from a comparable host cell by contacting the comparable host cell with a non-sugar-based detergent (e.g., Triton X-100).

In some embodiments, the contacting of the host cell with the sugar-based detergent decreases aggregation of the plurality of viruses or viral particles released from the host cell compared to other methods not using the sugar-based detergent. For example, the contacting of the host cell with the sugar-based detergent decreases aggregation of the plurality of viruses or viral particles released from the host cell compared to aggregation the same plurality of viruses or viral particles released from a comparable host cell by contacting the comparable host cell with a non-sugar-based detergent (e.g., Triton X-100). In some embodiments, the contacting of the host cell with the sugar-based detergent decreases aggregation of the plurality of viruses or viral particles released from the host cell by at least 0.1 fold, at least 0.2 fold, at least 0.4 fold, at least 0.8 fold, at least 1.0 fold, at least 1.2 fold, at least 1.5 fold, at least 2.0 fold, at least 3.0 fold, at least 4.0 fold, at least 5.0 fold, at least 10.0 fold, at least 20.0 fold, at least 50.0 fold, at least 100.0 fold, or more fold compared to aggregation of the same plurality of viruses or viral particles released from a comparable host cell by contacting the comparable host cell with a non-sugar-based detergent (e.g., Triton X-100).

In some embodiments, the contacting of the host cell with the sugar-based detergent increases infectivity of the plurality of viruses or viral particles released from the host cell compared to other methods not using the sugar-based detergent. For example, the contacting of the host cell with the sugar-based detergent increases infectivity of the plurality of viruses or viral particles released from the host cell compared to infectivity of the same plurality of viruses or viral particles released from a comparable host cell by contacting the comparable host cell with a non-sugar-based detergent (e.g., Triton X-100). In some embodiments, the contacting of the host cell with the sugar-based detergent increases infectivity of the plurality of viruses or viral particles released from the host cell by at least 0.1 fold, at least 0.2 fold, at least 0.4 fold, at least 0.8 fold, at least 1.0 fold, at least 1.2 fold, at least 1.5 fold, at least 2.0 fold, at least 3.0 fold, at least 4.0 fold, at least 5.0 fold, at least 10.0 fold, at least 20.0 fold, at least 50.0 fold, at least 100.0 fold, or more fold compared to infectivity of the same plurality of viruses or viral particles released from a comparable host cell by contacting the comparable host cell with a non-sugar-based detergent (e.g., Triton X-100).

In some embodiments, the contacting of the host cell with the sugar-based detergent decreases oxidative damage to the plurality of viruses or viral particles released from the host cell compared to other methods not using the sugar-based detergent. For example, the contacting of the host cell with the sugar-based detergent decreases oxidative damage of the plurality of viruses or viral particles released from the host cell compared to oxidative damage of the same plurality of viruses or viral particles released from a comparable host cell by contacting the comparable host cell with a non-sugar-based detergent (e.g., Triton X-100). In some embodiments, the contacting of the host cell with the sugar-based detergent decreases oxidative damage of the plurality of viruses or viral particles released from the host cell by at least 0.1 fold, at least 0.2 fold, at least 0.4 fold, at least 0.8 fold, at least 1.0 fold, at least 1.2 fold, at least 1.5 fold, at least 2.0 fold, at least 3.0 fold, at least 4.0 fold, at least 5.0 fold, at least 10.0 fold, at least 20.0 fold, at least 50.0 fold, at least 100.0 fold, or more fold compared to oxidative damage of the same plurality of viruses or viral particles released from a comparable host cell by contacting the comparable host cell with a non-sugar-based detergent (e.g., Triton X-100).

In some embodiments, the contacting of the host cell with the sugar-based detergent releases the plurality of viruses or viral particles encoded by a vector described herein (e.g., an AAV vector), while the sugar-based detergent also inactivates viruses that are different from the plurality of viruses or viral particles. For example, the sugar-based detergent can inactivate other viruses (e.g., baculovirus) that are not encoded by a vector described herein (e.g., an AAV vector). In some embodiments, the sugar-based detergent inactivates viruses not encoded by a vector described herein at a comparable level as viral inactivation by contacting the viruses not encoded by the vector with a formulation comprising a pH of 6, pH of 5, pH of 4, pH of 3, pH of 2, or pH of 1. In some embodiments, the sugar-based detergent inactivates viruses not encoded by a vector described herein at a comparable level as viral inactivation by contacting the viruses not encoded by the vector with a formulation comprising a pH of 3. In some embodiments, the sugar-based detergent, in combination with a formulation comprising a low pH (e.g., pH of 3), inactivates viruses not encoded by a vector without decreasing infectivity of the plurality of viruses or viral particles encoded by a vector described herein (e.g., an AAV vector). In some embodiments, the sugar-based detergent, in combination with a formulation comprising a low pH (e.g., pH of 3), inactivates viruses not encoded by a vector without increasing aggregation of the plurality of viruses or viral particles encoded by a vector described herein (e.g., an AAV vector). In some embodiments, the sugar-based detergent, in combination with a formulation comprising a low pH (e.g., pH of 3), inactivates viruses not encoded by a vector without decreasing oxidative damage of the plurality of viruses or viral particles encoded by a vector described herein (e.g., an AAV vector).

In some embodiments, described herein is a formulation comprising the sugar-based detergent described herein for contacting with the host cell and releasing the plurality of viruses or viral particles. In some embodiments, the formulation comprises at least one additional ingredient. In some embodiments, the at least one additional ingredient is a surfactant. In some embodiments, the at least one additional ingredient is a non-ionic surfactant. In some embodiments, the at least one additional ingredient is a non-ionic detergent (e.g., Tween 20). In some embodiments, the at least one additional ingredient is an anionic surfactant. In some embodiments, the at least one additional ingredient is a zwitterionic detergent. In some embodiments, the at least one additional ingredient is a detergent such as sodium lauroyl sarcosinate (SLS). In some embodiments, the formulation comprises at least one additional detergent such as Triton X-100. In some embodiments, the formulation does not include any additional detergent. For example, the formulation does not include Triton X-100 nor SLS. In some embodiments, the formulation comprises at least one additional ingredient that is a salt. For example, the formulation can include sodium chloride (NaCl) at 0.1 M NaCl, at 0.2 M NaCl, at 0.5 M NaCl, at 1.0 M NaCl, at 2.0 M NaCl, at 3.0 M NaCl, at 5.0 M NaCl, or at 10.0 M NaCl.

### Vector

Described herein, in some aspects, is vector for encoding the plurality of viruses or viral particles. In some embodiments, the vector is introduced in a host cell for expressing and manufacturing the plurality of viruses or viral particles. In some embodiments, the vector comprises engineered polynucleotide for encoding a therapeutic such as a peptide or a fusion protein. For examples, the engineered polynucleotide can encode a natriuretic peptide or a VEGF inhibitor (e.g., VEGF-Trap). In some embodiments, the vector comprises a viral vector such as an AAV vector comprising one or more expression cassettes for encoding and expressing one or more therapeutics. In some embodiments, the engineered polynucleotide comprises a vector. In some embodiments, the vector is a viral vector. In some embodiments, the engineered polynucleotide comprises an AAV vector. In some embodiments, the engineered polynucleotide comprises an AAV vector encoding an engineered AAV capsid. In some embodiments, the vector is a viral vector comprises an AAV vector. In some embodiments, the AAV vector comprises an AAV serotype comprising AAV1, AAV2, AAV3, AAV4, AAV5, AAV6, AAV7, AAV8, AAV9, AAV10, AAV11, AAV12, or any combination thereof. In some embodiments, the engineered polynucleotide is an AAV vector comprising the AAV2 serotype.

In some cases, the vector comprises additional features. Additional features can comprise sequences such as tags, signal peptides, intronic sequences, promoters, stuffer sequences, and the like. In some cases, the vector encodes a signal peptide. A signal peptide is sometimes referred to as signal sequence, targeting signal, localization signal, localization sequence, transit peptide, leader sequence or leader peptide, is a short peptide present at the N-terminus of the majority of newly synthesized proteins that are destined toward the secretory pathway. These proteins include those that reside either inside certain organelles (the endoplasmic reticulum, Golgi or endosomes), secreted from the cell, or inserted into most cellular membranes. In some cases, nucleic acids provided herein can comprise signal peptides. A signal peptide can be of any length but typically from 15-30 amino acids long. A signal peptide can be from about: 10-15, 10-20, 10-30, 15-20, 15-25, 15-30, 20-30, or 25-30 amino acids long.

In some cases, the vector comprises an intronic sequence. An intron is any nucleotide sequence within a sequence that can be removed by RNA splicing during maturation of the final RNA product. In other words, introns are non-coding regions of an RNA transcript, or the DNA encoding it, that are eliminated by splicing before translation. While introns do not encode protein products, they are players in gene expression regulation. Some introns themselves encode functional RNAs through further processing after splicing to generate noncoding RNA molecules. Alternative splicing is widely used to generate multiple proteins from a single gene. Furthermore, some introns play essential roles in a wide range of gene expression regulatory functions such as nonsense-mediated decay and mRNA export. In an embodiment, an intronic sequence is included in a nucleic acid of the disclosure and can be selected from: hCMV intron A, adenovirus tripartite leader sequence intron, SV40 intron, hamster EF-1 alpha gene intron 1, intervening sequence intron, human growth hormone intron, and/or human beta globin intron. Any number of intronic sequences are contemplated. In an embodiment, the intronic sequence is SV40. In some cases, at least 1, at least 2, at least 3, at least 4, at least 5, at least 6, at least 7, at least 8, at least 9, or up to 10 intronic sequences can be included in a nucleic acid.

In an embodiment, the vector comprises an additional feature including a promoter. Promoters are sequences of DNA to which proteins bind that initiate transcription of a single RNA from the DNA downstream of it. This RNA may encode a protein, or can have a function in and of itself, such as tRNA, mRNA, or rRNA. Promoters are located near the transcription start sites of genes, upstream on the DNA (towards the 5′ region of the sense strand). Promoters can be about 100-1000 base pairs long. Various promoters are contemplated and can be employed in the engineered polynucleotides of the disclosure. In an embodiment, a promoter is: a cytomegalovirus (CMV) promoter, an elongation factor 1 alpha (EF1α) promoter, a simian vacuolating virus (SV40) promoter, a phosphoglycerate kinase (PGK1) promoter, a ubiquitin C (Ubc) promoter, a human beta actin promoter, a CAG promoter, a Tetracycline response element (TRE) promoter, a UAS promoter, an Actin 5c (Ac5) promoter, a polyhedron promoter, a Ca2+/calmodulin-dependent protein kinase II (CaMKIIa) promoter, a GALI promoter, a GAL 10 promoter, a TEF1 promoter, a glyceraldehyde 3-phosphage dehydrogenase (GDS) promoter, an ADHI promoter, a CaMV35S promoter, a Ubi promoter, a human polymerase III RNA (H1) promoter, a U6 promoter, a polyadenylated construct thereof, and any combination thereof. In some cases, the promoter is the CMV promoter.

Any of the provided vector can comprise viral vector sequences. A viral vector can be, without limitation, a lentivirus, a retrovirus, or an adeno-associated virus. A viral vector can be an adeno-associated viral (AAV) vector. In some cases, a viral vector is an adeno-associated viral vector. Many serotypes of AAV vectors are contemplated and include but are not limited to: AAV1, AAV2, AAV3, AAV4, AAV5, AAV6, AAV7, AAV8, AAV9, AAV10, AAV11, and/or AAV12. Based on these initial serotypes, AAV capsid of each serotype can be engineered to make them better suited for biological functions, tissue or cell selection. In some embodiments, an AAV vector is AAV2 and variants AAV2.N53 and AAV2.N54. Chimeric AAV vectors are also contemplated that may contain at least 2 AAV serotypes. In some cases, at least 3, at least 4, at least 5, at least 6, at least 7, or up to 8 different serotypes are combined in a chimeric AAV vector. In some cases, only a portion of the AAV is chimeric. For example, suitable portions can include the capsid, VP1, VP2, or VP3 domains and/or Rep. In some cases, at least one of VP1, VP2, and VP3 has at least one amino acid substitution compared to an otherwise comparable wild-type AAV capsid protein. In some cases, a mutation can occur in VP1 and VP2, in VP1 and VP3, in VP2 and VP3, or in VP1, VP2, and VP3. In some embodiments, at least one of VP1, VP2, and VP3 has from one to about 25 amino acid substitutions compared to wild-type AAV VP1, VP2, and VP3, e.g., from about one to about 5, from about 5 to about 10, from about 10 to about 15, from about 15 to about 20, or from about 20 to about 25 amino acid substitutions compared to wild-type AAV VP1, VP2, and VP3. In some cases, a VP can be removed. For example, in some embodiments a mutant AAV does not comprise at least one of VP1, VP2, or VP3.

In some cases, an AAV vector can be modified. For example, an AAV vector can comprise a modification such as an insertion, deletion, chemical alteration, or synthetic modification. In some cases, a single nucleotide is inserted into an AAV vector. In other cases, multiple nucleotides are inserted into a vector described herein.

### Codon Optimization

In an embodiment, the vector described herein comprises a modification that confers enhanced expression of a biologic such as a VEGF inhibitor. For example, some biologics are derived from natural gene sequences and contain unmodified sequences that are not optimized for introduction and expression in target cells. In an embodiment, an isolated, engineered polynucleotide is codon optimized. Codon optimization can be specific for cell type-specific codon usage. Different organisms and cell types exhibit bias towards use of certain codons over others for the same amino acid. Some species are known to avoid certain codons almost entirely. Similarly, certain cell types are biased toward use of certain codons over others for the same amino acid. In an embodiment, a method of optimizing a codon of a engineered polynucleotide can comprise reassigning codon usage based on the frequencies of each codon's usage in a target cell. In some cases, a target cell can be of a certain tissue or organ. In some cases, a modification is performed to increase guanine and/or cytosine content.

In an embodiment, a vector can be modified to replace at least one codon with another codon coding for an identical amino acid. In some cases, a codon is modified within a coding region of a sequence. In some cases, a codon is modified within a non-coding region of a sequence. In some cases, a codon is modified within about 100, about 50, about 25, about 15, or about 5 bases from a termination codon. E-CAI can be utilized to estimate a value of a codon adaptation index.

Various modifications are contemplated herein. In some cases, codons can be interchanged. For example, a sequence can be modified to replace AGA with AGG. In other cases, CCC is replaced with CCT. In other cases, AGC is replaced with TCC. In other cases, CCC is replaced with CCG. Any of the non-limiting replacements provided in Table 25 can be applied to modify a nucleic acid. Any number of codons can be interchanged in a nucleic acid. In some cases, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 32, at least 34, at least 36, at least 38, at least 40, at least 42, at least 44, at least 46, at least 48, or up to 50 codons can be replaced. In an embodiment, an engineered polynucleotide comprises 3 codon modifications. In an embodiment, an engineered polynucleotide comprises 16 codon modifications. In an embodiment, an engineered polynucleotide comprises 3-5, 5-10, 5-15, 10-15, 10-20, 15-20, 1-20, 12-20, 12-25, 15-30, or 15-25 codon modifications. In an embodiment, an engineered polynucleotide comprises two codon modifications that are: AGA to AGG and at least one of: CCT to CCC, AGC to TCC, or CCC to CCG. In an embodiment, an engineered polynucleotide comprises three codon modifications that are: AGA to AGG and at least two of: CCT to CCC, AGC to TCC, or CCC to CCG. In an embodiment, an engineered polynucleotide comprises four codon modifications that are: AGA to AGG, CCT to CCC, AGC to TCC, and CCC to CCG. Additional modifications can comprise any of the codon modifications provided in Table 25 in combination with any of the above codons and/or any additional modifications possible from Table 25. In an embodiment, a nucleic acid is modified such that AGA is replaced with AGG and CCT is replaced with CCC. In an embodiment, a nucleic acid is modified such that AGA is replaced with AGG and AGC is replaced with TCC. In an embodiment, a nucleic acid is modified such that AGA is replaced with AGG and CCC is replaced with CCG.

TABLE 25
Non-limiting, exemplary codons that can be interchanged for modification of
nucleic acids. Thymine can be replaced with uracil in the below codons.

| AA | Codons | AA | Codons |
|---|---|---|---|
| Ala | GCT, GCC, GCA, GCG | Leu | TTA, TTG, CTT, CTC, CTA, CTG |
| Arg | CGT, CGC, CGA, CGG, AGA, AGG | Lys | AAA, AAG |
| Asn | AAT, AAC | Met | ATG |
| Asp | GAT, GAC | Phe | TTT, TTC |
| Cys | TGT, TGC | Pro | CCT, CCC, CCA, CCU |
| Gln | CAA, CAG | Ser | TCT, TCC, TCA, TCG, AGT, AGC |
| Glu | GAA, GAG | Thr | ACT, ACC, ACA, ACG |
| Gly | GGT, GGC, GGA, GGG | Trp | TGG |
| His | CAT, CAC | Tyr | TAT, TAC |
| Ile | ATT, ATC, ATA | Val | GTT, GTC, GTA, GTG |
| Start | ATG | Stop | TAA, TGA, TAG |

In some embodiments, a engineered polynucleotide sequence can comprise a viral vector sequence. In some embodiments, a viral vector sequence can be a scAAV vector sequence. In some embodiments, a AAV vector sequence can be of serotype AAV1, AAV2, AAV3, AAV4, AAV5, AAV6, AAV7, AAV8, AAV9, AAV10, AAV11, AAV12, or any combination thereof. In some embodiments, an AAV vector sequence can be of the AAV2 serotype. In some embodiments, a viral vector sequence can comprise sequences of at least 2 AAV serotypes. In some embodiments, at least two serotypes can be selected from AAV1, AAV2, AAV3, AAV4, AAV5, AAV6, AAV8, AAV9, AAV11, and AAV12.

In some cases, a modification can also comprise a chemical modification. Modified nucleic acids can comprise modifications of their backbones, sugars, or nucleobases, and even novel bases or base pairs. Modified nucleic acids can have improved chemical and/or biological stability. Decoration with diverse chemical substituents (e.g., hydrophobic groups) can also yield improved properties and functionalities such as new structural motifs and enhanced target binding.

Exemplary chemical modification include but are not limited to: 2′F, 2′-fluoro; 2′OMe, 2′-O-methyl; LNA, locked nucleic acid; FANA, 2′-fluoro arabinose nucleic acid; HNA, hexitol nucleic acid; 2′MOE, 2′-O-methoxyethyl; ribuloNA, (1′-3′)-β-L-ribulo nucleic acid; TNA, α-L-threose nucleic acid; tPhoNA, 3′-2′ phosphonomethyl-threosyl nucleic acid; dXNA, 2′-deoxyxylonucleic acid; PS, phosphorothioate; phNA, alkyl phosphonate nucleic acid; PNA, and peptide nucleic acid.

### Engineered Capsid

In some aspects, the vector described herein encodes an engineered capsid. In some embodiments, the engineered capsid is a modified AAV capsid. A modified AAV capsid can comprise exogenous sequences as compared to an otherwise comparable unmodified AAV capsid. Exogenous sequences can refer to exogenous polypeptide sequences. AAV capsids can be modified to confer upon them, and any compositions and/or methods in which they are utilized, improved functionality thereby resulting in better therapeutics, particularly for ocular use.

The AAV wild-type (WT) genome contains at least three genes: rep, cap, and X. The X gene is located at the 3′ end of the genome (nucleotides 3929-4393 in AAV2) and seems to code for a protein with supportive function in genome replication. Significantly more information is available for rep and cap. The rep gene is located in the first half of the AAV WT genome and codes for a family of non-structural proteins (Rep proteins) required for viral transcription control and replication as well as packaging of viral genomes into the newly produced, pre-assembled capsids. The second half of the AAV genome contains the cap gene, which codes for the viral proteins (VPs) VP1, VP2, and VP3, and the assembly-activating protein (AAP). Transcription of all VPs, which are the capsid monomers, is controlled by a single promoter (p40 in case of AAV2) resulting in a single mRNA. Splicing (VP1) and an unusual translational start codon (VP2) are responsible for an approximately 10 times lower presence of VP1 and VP2 compared with VP3. When encoded by a single gene, AAV VPs share most of their amino acids. Specifically, the entire VP3 sequence is also contained within VP2 and VP1 (“common VP3 region”), and also VP2 and VP1 share approximately 65 amino acids (“common VP1/VP2 region”). Only VP1 contains a unique sequence at its N terminus (approximately 138 amino acids, VP1 unique). AAP was identified in 2010 as a 23 kD protein encoded in an alternative cap ORF. It is used for stabilizing and transporting newly produced VP proteins from the cytoplasm into the cell nucleus. While AAV serotypes 1-3, 6-9, and rh10 failed to produce capsids in the absence of AAP, a low but detectable capsid production was reported for AAV4 and AAV5.

In an aspect, an AAV can comprise a modification. A modification can be of a rep, cap, and/or X coding polypeptide sequence of an AAV. In some cases, the modification can be of a cap polypeptide. A cap polypeptide can be modified in any one of the VP domains, for example VP1, VP2, and/or VP3. In some cases, VP1 is modified. In some cases, VP2 is modified. In some cases, VP3 is modified. In some aspects, two or all of the VP domains can be modified. In some cases, VP1 and VP2 are modified. In some cases, VP1 and VP3 are modified. Additionally, VP2 and VP3 can be modified or VP1, VP2, and VP3 are modified. Other combinations are contemplated, such as modifications in Rep and Cap, Cap and X, Rep and X, and/or Rep, Cap, and X. Any combination of domains can be modified such as any one of the aforementioned VP modifications in conjunction with a Rep and/or X modification. In some cases, Rep and VP1 and/or VP2 are modified. In some aspects, a subject Rep is modified. A rep modification can comprise a modification as provided herein and can be in at least one of Rep 78, Rep 68, Rep 52 or Rep 40. In some cases, a Rep is of a different AAV serotype than a subject capsid.

In some cases, a modification is of an AAV capsid. Capsids of AAV serotypes are assembled from 60 VP monomers with approximately 50 copies of VP3, 5 copies of VP2, and 5 copies of VP1. Topological prominent capsid surface structures are pores or “channel-like-structures” at each fivefold, depressions at each twofold, and three protrusions surrounding each threefold axis of symmetry. The pores allow exchange between the capsid interior and the outside. The depressions, more precisely the floor at each twofold axis, are the thinnest part of the viral capsid. The protrusions around the threefold axis harbor five of the nine so-called variable regions (VRs). Specifically, VR-IV, -V, and -VIII form loops (loop 1-4) at the top of the protrusions, while VR-VI and -VII are found at their base. VRs differ between serotypes and are responsible for serotype-specific variations in antibody and receptor binding. Because of their exposed positions and their function in receptor binding, VRs forming loops of the protrusions are ideal positions for capsid modifications aiming to re-direct or expand AAV tropism (cell surface targeting). While a re-directed tropism (vector re-targeting) combines ablation of natural receptor binding, for example by site-directed mutagenesis, with insertion of a ligand that mediates transduction through a novel non-natural AAV receptor, AAV vectors with tropism expansion gain the ability to transduce cells through an extra receptor while maintaining their natural receptor binding abilities.

In some aspects, a modification of an AAV capsid, can refer to an insertion of an exogenous polypeptide sequence. In other aspects, a modification can refer to a deletion in a polypeptide sequence. A modification can also refer to a modification of at least one amino acid residue, canonical or non-canonical, in a polypeptide sequence.

An insertion can comprise inserting at least 1 exogenous amino acid residue into a sequence coding an AAV capsid. The amino acid can refer to a canonical amino acid or a non-canonical amino acid. Any number of amino acid residues can be inserted. In some cases, an insertion site can be in the GH loop, or loop IV, of the AAV capsid protein, e.g., in a solvent-accessible portion of the GH loop, or loop IV, of the AAV capsid protein.

In some cases, a modification comprises insertion of an exogenous polypeptide sequence that comprises a sequence of Formula 1: X0-X1-X2-X1-X3-X1-X1-X4. In some cases, X0 is Valine (V), Isoleucine (I), Leucine (L), Phenylalanine (F), Tryptophan (W), Tyrosine (Y) or Methionine (M). In some cases, X1 is Alanine (A), Asparagine (N), Glutamine (Q), Serine(S), Threonine (T), Glutamic Acid (E), Aspartic Acid (D), Lysine (K), Arginine (R), or Histidine (H). In some cases, X2 is V, I, L, or M, where X3 is E, S, or Q. In some cases, X4 is K, R, E, or A. In some cases, Formula 1 further comprises X5. X5 can be Proline (P) or R.

In some cases, Formula 1 comprises: L-A-L-G-X3-X1-X1-X4 (SEQ ID NO: 42), L-K-L-G-X3-X1-X1-X4 (SEQ ID NO: 43), or V-K-L-G-X3-X1-X1-X4 (SEQ ID NO: 44). In some cases, Formula 1 comprises V-K-L-G-X3-X1-X1-X4 (SEQ ID NO: 45). In some cases, an exogenous polypeptide is V-K-L-G-X3-X1-T-X4 (SEQ ID NO: 46) and/or V-K-L-G-X3-X1-X1-K (SEQ ID NO: 47). In some cases, an exogenous polypeptide comprises L-A-L-G-X3-X1-X1-X4 (SEQ ID NO: 48). In some cases, an exogenous polypeptide comprises L-A-L-G-X3-X1-T-X4 (SEQ ID NO: 49) and/or L-A-L-G-X3-X1-S-X4 (SEQ ID NO: 50). In some cases, an exogenous polypeptide comprises: L-A-L-G-X3-X1-T-R (SEQ ID NO: 51), L-A-L-G-X3-X1-T-K (SEQ ID NO: 52), L-A-L-G-X3-X1-T-E (SEQ ID NO: 53), and/or L-A-L-G-X3-X1-T-A (SEQ ID NO: 54). In some cases, an exogenous polypeptide comprises L-A-L-G-X3-X1-S-K (SEQ ID NO: 56). In some cases, an exogenous polypeptide comprises L-K-L-G-X3-X1-X1-X4 (SEQ ID NO: 57). In some cases, an exogenous polypeptide comprises: L-K-L-G-X3-X1-T-X4 (SEQ ID NO: 58). In some cases, an exogenous polypeptide comprises: L-K-L-G-X3-X1-T-K (SEQ ID NO: 59).

In some cases, an exogenous polypeptide comprises a sequence of Formula 1. In some cases, a sequence of Formula I comprises a polypeptide sequence having at least 60%, 62%, 64%, 66%, 68%, 70%, 72%, 74%, 76%, 78%, 80%, 82%, 84%, 86%, 88%, 90%, 92%, 94%, 96%, 98%, or up to about 100% identity with a sequence of Table 26. In some cases, an exogenous polypeptide is one of Table 26 with 0-2 modifications to a residue.

In some cases, at least 2 of the exogenous polypeptides, such as those described by Formula 1, are inserted into a capsid sequence of an AAV provided herein. The at least 2 exogenous polypeptides can be inserted into the same location or at different locations. In an aspect, any one of the exogenous polypeptide sequences provided in Table 27 can be inserted into an unmodified AAV capsid sequence, such as those wildtype sequences provided in Table 27, to generate modified AAV capsid.

TABLE 26
Exemplary exogenous polypeptide sequences (SEQ ID NOs: 61-82) that can be inserted into
AAV capsids (exemplary insertion sites are shown for AAV2 but comparable locations of
other AAV serotypes are also contemplated) and exemplary modified AAV2 capsid
sequences (SEQ ID NOs: 91-110).

| SEQ ID | | Modified Capsid Region | |
|---|---|---|---|
| NO | Clone No. | of AAV2 | Exogenous Polypeptide Sequence |
| 61 | V466 | 453 | LALGETTRPA |
| 62 | V467 | 587 | LALGETTKPA |
| 63 | V468 | 452 | ALGETTKP |
| 64 | V471 | 452 | ALGETTKP |
| 65 | V471 | 587 | LALGETTKPA |
| 66 | AMI051 | 587 | LKLGQTTKPK |
| 67 | AMI052 | 587 | LALGQTTKPK |
| 68 | AMI053 | 587 | LKLGQTTKPA |
| 69 | AMI054 | 587 | LALGQTTKPA |
| 70 | AMI097 | 453 | LALGQTTKPA |
| 71 | AMI098 | 587 | LALGQTTEPA |
| 72 | AMI099 | 588 | LALGQTTKPA |
| 73 | AMI100 | 585 | LALGQTTKPA |
| 74 | AMI101 | 587 | VALGQTTKPA |
| 75 | AMI102 | 586 | LALGESTARG |
| 76 | AMI103 | 586 | LALGETSKRA |
| 77 | AMI104 | 586 | LALGQSTKPA |
| 78 | AMI105 | 452 | LALGQTTKPA |
| 79 | AMI106 | 453 | LALGQTTKPA |
| 80 | AMI106 | 587 | LALGQTTKPA |
| 81 | AMI107 | 587 | LALGQTTKPALALGQTTKPA |
| 82 | AMI110 | 587 | VKLGQTTKPA |

| SEQ ID | | |
|---|---|---|
| NO | Clone | Modified AAV2 Capsid Sequence |
| 91 | V466 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGLALGETTRPATTTQSRLQFSQAGAS |
| | | DIRDQSRNWLPGPCYRQQRVSKTSADNNNSEYSWTGATK |
| | | YHLNGRDSLVNPGPAMASHKDDEEKFFPQSGVLIFGKQGS |
| | | EKTNVDIEKVMITDEEEIRTTNPVATEQYGSVSTNLQRGNR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 92 | V467 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLALGETTKPAR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 93 | V468 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSALGETTKPGTTTQSRLQFSQAGASDIR |
| | | DQSRNWLPGPCYRQQRVSKTSADNNNSEYSWTGATKYHL |
| | | NGRDSLVNPGPAMASHKDDEEKFFPQSGVLIFGKQGSEKT |
| | | NVDIEKVMITDEEEIRTTNPVATEQYGSVSTNLQRGNRQAA |
| | | TADVNTQGVLPGMVWQDRDVYLQGPIWAKIPHTDGHFHP |
| | | SPLMGGFGLKHPPPQILIKNTPVPANPSTTFSAAKFASFITQY |
| | | STGQVSVEIEWELQKENSKRWNPEIQYTSNYNKSVNVDFT |
| | | VDTNGVYSEPRPIGTRYLTRNL |
| 94 | V471ª | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSALGETTKPGTTTQSRLQFSQAGASDIR |
| | | DQSRNWLPGPCYRQQRVSKTSADNNNSEYSWTGATKYHL |
| | | NGRDSLVNPGPAMASHKDDEEKFFPQSGVLIFGKQGSEKT |
| | | NVDIEKVMITDEEEIRTTNPVATEQYGSVSTNLQRGNLALG |
| | | ETTKPARQAATADVNTQGVLPGMVWQDRDVYLQGPIWA |
| | | KIPHTDGHFHPSPLMGGFGLKHPPPQILIKNTPVPANPSTTFS |
| | | AAKFASFITQYSTGQVSVEIEWELQKENSKRWNPEIQYTSN |
| | | YNKSVNVDFTVDTNGVYSEPRPIGTRYLTRNL |
| 95 | AMI051 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLKLGQTTKPKR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 96 | AMI052 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLALGQTTKPKR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 97 | AMI053 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLKLGQTTKPAR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 98 | AMI054 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLALGQTTKPAR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 99 | AMI097 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGLALGQTTKPATTTQSRLQFSQAGAS |
| | | DIRDQSRNWLPGPCYRQQRVSKTSADNNNSEYSWTGATK |
| | | YHLNGRDSLVNPGPAMASHKDDEEKFFPQSGVLIFGKQGS |
| | | EKTNVDIEKVMITDEEEIRTTNPVATEQYGSVSTNLQRGNR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 100 | AMI098 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLALGQTTEPAR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 101 | AMI099 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNRLALGQTTKPA |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 102 | AMI100 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRLALGQTTKPAGNR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 103 | AMI101 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNVALGQTTKPAR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 104 | AMI102 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGLALGESTARGNR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 105 | AMI103 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGLALGETSKRANR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 106 | AMI104 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGLALGQSTKPANR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 107 | AMI105 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSLALGQTTKPAGTTTQSRLQFSQAGAS |
| | | DIRDQSRNWLPGPCYRQQRVSKTSADNNNSEYSWTGATK |
| | | YHLNGRDSLVNPGPAMASHKDDEEKFFPQSGVLIFGKQGS |
| | | EKTNVDIEKVMITDEEEIRTTNPVATEQYGSVSTNLQRGNR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 108 | AMI106 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGLALGQTTKPATTTQSRLQFSQAGAS |
| | | DIRDQSRNWLPGPCYRQQRVSKTSADNNNSEYSWTGATK |
| | | YHLNGRDSLVNPGPAMASHKDDEEKFFPQSGVLIFGKQGS |
| | | EKTNVDIEKVMITDEEEIRTTNPVATEQYGSVSTNLQRGNL |
| | | ALGQTTKPARQAATADVNTQGVLPGMVWQDRDVYLQGPI |
| | | WAKIPHTDGHFHPSPLMGGFGLKHPPPQILIKNTPVPANPST |
| | | TFSAAKFASFITQYSTGQVSVEIEWELQKENSKRWNPEIQY |
| | | TSNYNKSVNVDFTVDTNGVYSEPRPIGTRYLTRNL |
| 109 | AMI107 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLALGQTTKPAL |
| | | ALGQTTKPARQAATADVNTQGVLPGMVWQDRDVYLQGPI |
| | | WAKIPHTDGHFHPSPLMGGFGLKHPPPQILIKNTPVPANPST |
| | | TFSAAKFASFITQYSTGQVSVEIEWELQKENSKRWNPEIQY |
| | | TSNYNKSVNVDFTVDTNGVYSEPRPIGTRYLTRNL |
| 110 | AMI110 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNVAKGQTTKPA |
| | | RQAATADVNTQGVLPGMVWQDRDVYLQGPIWAKIPHTD |
| | | GHFHPSPLMGGFGLKHPPPQILIKNTPVPANPSTTFSAAKFA |
| | | SFITQYSTGQVSVEIEWELQKENSKRWNPEIQYTSNYNKSV |
| | | NVDFTVDTNGVYSEPRPIGTRYLTRNL |

TABLE 27
Exemplary wild type AAV capsid polypeptide sequences

| SEQ | AAV | |
|---|---|---|
| ID NO | Serotype | WT Polypeptide Sequence |
| 121 | AAV2 | MAADGYLPDWLEDTLSEGIRQWWKLKPGPPPPKPAERHKDDSR |
| | | GLVLPGYKYLGPFNGLDKGEPVNEADAAALEHDKAYDRQLDS |
| | | GDNPYLKYNHADAEFQERLKEDTSFGGNLGRAVFQAKKRVLEP |
| | | LGLVEEPVKTAPGKKRPVEHSPVEPDSSSGTGKAGQQPARKRLN |
| | | FGQTGDADSVPDPQPLGQPPAAPSGLGTNTMATGSGAPMADNN |
| | | EGADGVGNSSGNWHCDSTWMGDRVITTSTRTWALPTYNNHLY |
| | | KQISSQSGASNDNHYFGYSTPWGYFDFNRFHCHFSPRDWQRLIN |
| | | NNWGFRPKRLNFKLFNIQVKEVTQNDGTTTIANNLTSTVQVFTD |
| | | SEYQLPYVLGSAHQGCLPPFPADVFMVPQYGYLTLNNGSQAVG |
| | | RSSFYCLEYFPSQMLRTGNNFTFSYTFEDVPFHSSYAHSQSLDRL |
| | | MNPLIDQYLYYLSRTNTPSGTTTQSRLQFSQAGASDIRDQSRNW |
| | | LPGPCYRQQRVSKTSADNNNSEYSWTGATKYHLNGRDSLVNPG |
| | | PAMASHKDDEEKFFPQSGVLIFGKQGSEKTNVDIEKVMITDEEEI |
| | | RTTNPVATEQYGSVSTNLQRGNRQAATADVNTQGVLPGMVWQ |
| | | DRDVYLQGPIWAKIPHTDGHFHPSPLMGGFGLKHPPPQILIKNTP |
| | | VPANPSTTFSAAKFASFITQYSTGQVSVEIEWELQKENSKRWNPE |
| | | IQYTSNYNKSVNVDFTVDINGVYSEPRPIGTRYLTRNL |
| 122 | AAV4 | MTDGYLPDWLEDNLSEGVREWWALQPGAPKPKANQQHQDNA |
| | | RGLVLPGYKYLGPGNGLDKGEPVNAADAAALEHDKAYDQQLK |
| | | AGDNPYLKYNHADAEFQQRLQGDTSFGGNLGRAVFQAKKRVL |
| | | EPLGLVEQAGETAPGKKRPLIESPQQPDSSTGIGKKGKQPAKKK |
| | | LVFEDETGAGDGPPEGSTSGAMSDDSEMRAAAGGAAVEGGQG |
| | | ADGVGNASGDWHCDSTWSEGHVTTTSTRTWVLPTYNNHLYKR |
| | | LGESLQSNTYNGFSTPWGYFDFNRFHCHFSPRDWQRLINNNWG |
| | | MRPKAMRVKIFNIQVKEVTTSNGETTVANNLTSTVQIFADSSYE |
| | | LPYVMDAGQEGSLPPFPNDVFMVPQYGYCGLVTGNTSQQQTDR |
| | | NAFYCLEYFPSQMLRTGNNFEITYSFEKVPFHSMYAHSQSLDRL |
| | | MNPLIDQYLWGLQSTTTGTTLNAGTATTNFTKLRPTNFSNFKKN |
| | | WLPGPSIKQQGFSKTANQNYKIPATGSDSLIKYETHSTLDGRWS |
| | | ALTPGPPMATAGPADSKFSNSQLIFAGPKQNGNTATVPGTLIFTS |
| | | EEELAATNATDTDMWGNLPGGDQSNSNLPTVDRLTALGAVPG |
| | | MVWQNRDIYYQGPIWAKIPHTDGHFHPSPLIGGFGLKHPPPQIFI |
| | | KNTPVPANPATTFSSTPVNSFITQYSTGQVSVQIDWEIQKERSKR |
| | | WNPEVQFTSNYGQQNSLLWAPDAAGKYTEPRAIGTRYLTHHL |
| 123 | AAV5 | MSFVDHPPDWLEEVGEGLREFLGLEAGPPKPKPNQQHQDQARG |
| | | LVLPGYNYLGPGNGLDRGEPVNRADEVAREHDISYNEQLEAGD |
| | | NPYLKYNHADAEFQEKLADDTSFGGNLGKAVFQAKKRVLEPFG |
| | | LVEEGAKTAPTGKRIDDHFPKRKKARTEEDSKPSTSSDAEAGPS |
| | | GSQQLQIPAQPASSLGADTMSAGGGGPLGDNNQGADGVGNAS |
| | | GDWHCDSTWMGDRVVTKSTRTWVLPSYNNHQYREIKSGSVDG |
| | | SNANAYFGYSTPWGYFDFNRFHSHWSPRDWQRLINNYWGFRP |
| | | RSLRVKIFNIQVKEVTVQDSTTTIANNLTSTVQVFTDDDYQLPYV |
| | | VGNGTEGCLPAFPPQVFTLPQYGYATLNRDNTENPTERSSFFCLE |
| | | YFPSKMLRTGNNFEFTYNFEEVPFHSSFAPSQNLFKLANPLVDQ |
| | | YLYRFVSTNNTGGVQFNKNLAGRYANTYKNWFPGPMGRTQG |
| | | WNLGSGVNRASVSAFATTNRMELEGASYQVPPQPNGMTNNLQ |
| | | GSNTYALENTMIFNSQPANPGTTATYLEGNMLITSESETQPVNR |
| | | VAYNVGGQMATNNQSSTTAPATGTYNLQEIVPGSVWMERDVY |
| | | LQGPIWAKIPETGAHFHPSPAMGGFGLKHPPPMMLIKNTPVPGNI |
| | | TSFSDVPVSSFITQYSTGQVTVEMEWELKKENSKRWNPEIQYTN |
| | | NYNDPQFVDFAPDSTGEYRTTRPIGTRYLTRPL |
| 124 | AAV6 | MAADGYLPDWLEDNLSEGIREWWDLKPGAPKPKANQQKQDD |
| | | GRGLVLPGYKYLGPFNGLDKGEPVNAADAAALEHDKAYDQQL |
| | | KAGDNPYLRYNHADAEFQERLQEDTSFGGNLGRAVFQAKKRV |
| | | LEPFGLVEEGAKTAPGKKRPVEQSPQEPDSSSGIGKTGQQPAKK |
| | | RLNFGQTGDSESVPDPQPLGEPPATPAAVGPTTMASGGGAPMA |
| | | DNNEGADGVGNASGNWHCDSTWLGDRVITTSTRTWALPTYNN |
| | | HLYKQISSASTGASNDNHYFGYSTPWGYFDFNRFHCHFSPRDW |
| | | QRLINNNWGFRPKRLNFKLFNIQVKEVTTNDGVTTIANNLTSTV |
| | | QVFSDSEYQLPYVLGSAHQGCLPPFPADVFMIPQYGYLTLNNGS |
| | | QAVGRSSFYCLEYFPSQMLRTGNNFTFSYTFEDVPFHSSYAHSQ |
| | | SLDRLMNPLIDQYLYYLNRTQNQSGSAQNKDLLFSRGSPAGMS |
| | | VQPKNWLPGPCYRQQRVSKTKTDNNNSNFTWTGASKYNLNGR |
| | | ESIINPGTAMASHKDDKDKFFPMSGVMIFGKESAGASNTALDNV |
| | | MITDEEEIKATNPVATERFGTVAVNLQSSSTDPATGDVHVMGAL |
| | | PGMVWQDRDVYLQGPIWAKIPHTDGHFHPSPLMGGFGLKHPPP |
| | | QILIKNTPVPANPPAEFSATKFASFITQYSTGQVSVEIEWELQKEN |
| | | SKRWNPEVQYTSNYAKSANVDFTVDNNGLYTEPRPIGTRYLTR |
| | | PL |
| 125 | AAV11 | MAADGYLPDWLEDNLSEGIREWWDLKPGAPKPKANQQKQDD |
| | | GRGLVLPGYKYLGPFNGLDKGEPVNAADAAALEHDKAYDQQL |
| | | KAGDNPYLRYNHADAEFQERLQEDTSFGGNLGRAVFQAKKRV |
| | | LEPLGLVEEGAKTAPGKKRPLESPQEPDSSSGIGKKGKQPARKR |
| | | LNFEEDTGAGDGPPEGSDTSAMSSDIEMRAAPGGNAVDAGQGS |
| | | DGVGNASGDWHCDSTWSEGKVTTTSTRTWVLPTYNNHLYLRL |
| | | GTTSSSNTYNGFSTPWGYFDFNRFHCHFSPRDWQRLINNNWGL |
| | | RPKAMRVKIFNIQVKEVTTSNGETTVANNLTSTVQIFADSSYELP |
| | | YVMDAGQEGSLPPFPNDVFMVPQYGYCGIVTGENQNQTDRNAF |
| | | YCLEYFPSQMLRTGNNFEMAYNFEKVPFHSMYAHSQSLDRLMN |
| | | PLLDQYLWHLQSTTSGETLNQGNAATTFGKIRSGDFAFYRKNW |
| | | LPGPCVKQQRFSKTASQNYKIPASGGNALLKYDTHYTLNNRWS |
| | | NIAPGPPMATAGPSDGDFSNAQLIFPGPSVTGNTTTSANNLLFTS |
| | | EEEIAATNPRDTDMFGQIADNNQNATTAPITGNVTAMGVLPGM |
| | | VWQNRDIYYQGPIWAKIPHADGHFHPSPLIGGFGLKHPPPQIFIK |
| | | NTPVPANPATTFTAARVDSFITQYSTGQVAVQIEWEIEKERSKR |
| | | WNPEVQFTSNYGNQSSMLWAPDTTGKYTEPRVIGSRYLTNHL |
| 126 | AAV12 | MAADGYLPDWLEDNLSEGIREWWALKPGAPQPKANQQHQDN |
| | | GRGLVLPGYKYLGPFNGLDKGEPVNEADAAALEHDKAYDKQL |
| | | EQGDNPYLKYNHADAEFQQRLATDTSFGGNLGRAVFQAKKRIL |
| | | EPLGLVEEGVKTAPGKKRPLEKTPNRPTNPDSGKAPAKKKQKD |
| | | GEPADSARRTLDFEDSGAGDGPPEGSSSGEMSHDAEMRAAPGG |
| | | NAVEAGQGADGVGNASGDWHCDSTWSEGRVTTTSTRTWVLPT |
| | | YNNHLYLRIGTTANSNTYNGFSTPWGYFDFNRFHCHFSPRDWQ |
| | | RLINNNWGLRPKSMRVKIFNIQVKEVTTSNGETTVANNLTSTVQ |
| | | IFADSTYELPYVMDAGQEGSFPPFPNDVFMVPQYGYCGVVTGK |
| | | NQNQTDRNAFYCLEYFPSQMLRTGNNFEVSYQFEKVPFHSMYA |
| | | HSQSLDRMMNPLLDQYLWHLQSTTTGNSLNQGTATTTYGKITT |
| | | GDFAYYRKNWLPGACIKQQKFSKNANQNYKIPASGGDALLKY |
| | | DTHTTLNGRWSNMAPGPPMATAGAGDSDFSNSQLIFAGPNPSG |
| | | NTTTSSNNLLFTSEEEIATTNPRDTDMFGQIADNNQNATTAPHIA |
| | | NLDAMGIVPGMVWQNRDIYYQGPIWAKVPHTDGHFHPSPLMG |
| | | GFGLKHPPPQIFIKNTPVPANPNTTFSAARINSFLTQYSTGQVAV |
| | | QIDWEIQKEHSKRWNPEVQFTSNYGTQNSMLWAPDNAGNYHE |
| | | LRAIGSRFLTHHL |

Similarly, a deletion can comprise deleting at least 1 amino acid residue in a sequence that codes for an AAV capsid. Any number of amino acids can be deleted. In some cases, at least, or at most: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or up to about 50 exogenous amino acid residues can be inserted and/or deleted in a polypeptide sequence that codes for an AAV capsid. In some cases, at least or at most: 1-5, 5-10, 10-15, 15-20, or combinations thereof of exogenous amino acid residues can be inserted and/or deleted in a polypeptide sequence that codes for an AAV capsid. In some cases, from about or up to about: 5 amino acids to about 11 amino acids are inserted in an insertion site in the GH loop or loop IV of the capsid protein relative to a corresponding unmodified AAV capsid protein. For example, the insertion site can be between amino acids 587 and 588 of AAV2, or the corresponding positions of the capsid subunit of another AAV serotype. It should be noted that the insertion site 587-588 is based on an AAV2 capsid protein. From about 5 amino acids to about 11 amino acids can be inserted in a corresponding site in an AAV serotype other than AAV2 (e.g., AAV5, AAV6, AAV8, AAV9, etc.).

In some embodiments, the insertion site is a single insertion site between two adjacent amino acids located between amino acids 570-614 of VP1 of any AAV serotype, e.g., the insertion site is between two adjacent amino acids located in amino acids 570-610, amino acids 580-600, amino acids 570-575, amino acids 575-580, amino acids 580-585, amino acids 585-590, amino acids 590-600, or amino acids 600-614, of VP1 of any AAV serotype or variant. For example, the insertion site can be between amino acids 580 and 581, amino acids 581 and 582, amino acids 583 and 584, amino acids 584 and 585, amino acids 585 and 586, amino acids 586 and 587, amino acids 587 and 588, amino acids 588 and 589, or amino acids 589 and 590. The insertion site can be between amino acids 575 and 576, amino acids 576 and 577, amino acids 577 and 578, amino acids 578 and 579, or amino acids 579 and 580. The insertion site can be between amino acids 590 and 591, amino acids 591 and 592, amino acids 592 and 593, amino acids 593 and 594, amino acids 594 and 595, amino acids 595 and 596, amino acids 596 and 597, amino acids 597 and 598, amino acids 598 and 599, or amino acids 599 and 600.

In some aspects, an insertion site can be between amino acids 587 and 588 of AAV2, between amino acids 590 and 591 of AAV1, between amino acids 575 and 576 of AAV5, between amino acids 590 and 591 of AAV6, between amino acids 589 and 590 of AAV7, between amino acids 590 and 591 of AAV8, between amino acids 588 and 589 of AAV9, or between amino acids 588 and 589 of AAV10.

As another example, the insertion site can be between amino acids 450 and 460 of an AAV capsid protein, as shown in Table 26. For example, the insertion site can be at (e.g., immediately N-terminal to) amino acid 453 of AAV2, at amino acid 454 of AAV1, at amino acid 454 of AAV6, at amino acid 456 of AAV7, at amino acid 456 of AAV8, at amino acid 454 of AAV9, or at amino acid 456 of AAV10.

In some embodiments, a subject capsid protein includes a GH loop comprising an amino acid sequence having at least about 85%, at least about 90%, at least about 95%, at least about 98%, at least about 99%, or 100%, amino acid sequence identity to an amino acid sequence set forth in Table 26. Those skilled in the art would know, based on a comparison of the amino acid sequences of capsid proteins of various AAV serotypes, where an insertion site “corresponding to amino acids 587-588 of AAV2” would be in a capsid protein of any given AAV serotype.

In some cases, an exogenous polypeptide can have from 0 to 4 spacer amino acids (Y₁-Y₄) at the amino terminus and/or at the carboxyl terminus of any one of the exemplary polypeptides of Table 26 or Formula 1. Suitable spacer amino acids include, but are not limited to, leucine, alanine, glycine, and/or serine.

A modification of an AAV capsid can comprise a modification of at least one amino acid residue in a polypeptide sequence. In some cases, a modification can be made at any AAV capsid position, as described herein, and can include any number of modifications. In some cases, a modification can comprise a mutation. A mutation can comprise: a point mutation, missense mutation, nonsense mutation, deletion, duplication, frameshift, and/or repeat expansion.

In an aspect, an amino acid can be a non-polar, aliphatic residue such as glycine, alanine, valine, leucine, isoleucine, or proline. In an aspect, an amino acid residue is aromatic and is phenylalanine, tyrosine, or tryptophan. In an aspect, an amino acid residue is polar, non-charged and is serine, threonine, cysteine, methionine, asparagine, or glutamine. In an aspect, an amino acid is positively charged and is lysine, arginine, or histidine. In an aspect, an amino acid is negatively charged and is aspartate or glutamate.

In some cases, a mutation is a point mutation. A point mutation comprises a change from a charged amino acid residue to a polar or non-polar amino acid residue. In some cases, the charged amino acid is positively charged. In some cases, the charged amino acid is negatively charged.

A point mutation can be a conservative mutation. Non-limiting examples of conservative mutations comprise: a nonpolar aliphatic amino acid to a nonpolar aliphatic amino acid, a polar amino acid to a polar amino acid, a positively charged amino acid to a positively charged amino acid, a negatively charged amino acid to a negatively charged amino acid, and an aromatic amino acid to an aromatic amino acid. For example, 20 naturally occurring amino acids can share similar characteristics. Aliphatic amino acids can be: glycine, alanine, valine, leucine, or isoleucine. Hydroxyl or sulfur/selenium-containing amino acids can be: Serine, cysteine, selenocysteine, threonine, or methionine. A cyclic amino acid can be proline. An aromatic amino acid can be phenylalanine, tyrosine, or tryptophan. A basic amino acid can be histidine, lysine, and arginine. An acidic amino acid can be aspartate, glutamate, asparagine, or glutamine. A conservative mutation can be, serine to glycine, serine to alanine, serine to serine, serine to threonine, serine to proline. A conservative mutation can be arginine to asparagine, arginine to lysine, arginine to glutamine, arginine to arginine, arginine to histidine. A conservative mutation can be Leucine to phenylalanine, leucine to isoleucine, leucine to valine, leucine to leucine, leucine to methionine. A conservative mutation can be proline to glycine, proline to alanine, proline to serine, proline to threonine, proline to proline. A conservative mutation can be threonine to glycine, threonine to alanine, threonine to serine, threonine to threonine, threonine to proline. A conservative mutation can be alanine to glycine, alanine to threonine, alanine to proline, alanine to alanine, alanine to serine. A conservative mutation can be valine to methionine, valine to phenylalanine, valine to isoleucine, valine to leucine, valine to valine. A conservative mutation can be glycine to alanine, glycine to threonine, glycine to proline, glycine to serine, glycine to glycine. A conservative mutation can be Isoleucine to phenylalanine, isoleucine to isoleucine, isoleucine to valine, isoleucine to leucine, isoleucine to methionine. A conservative mutation can be phenylalanine to tryptophan, phenylalanine to phenylalanine, phenylalanine to tyrosine. A conservative mutation can be tyrosine to tryptophan, tyrosine to phenylalanine, tyrosine to tyrosine. A conservative mutation can be cysteine to serine, cysteine to threonine, cysteine to cysteine. A conservative mutation can be histidine to asparagine, histidine to lysine, histidine to glutamine, histidine to arginine, histidine to histidine. A conservative mutation can be glutamine to glutamic acid, glutamine to asparagine, glutamine to aspartic acid, glutamine to glutamine. A conservative mutation can be asparagine to glutamic acid, asparagine to asparagine, asparagine to aspartic acid, asparagine to glutamine. A conservative mutation can be lysine to asparagine, lysine to lysine, lysine to glutamine, lysine to arginine, lysine to histidine. A conservative mutation can be aspartic acid to glutamic acid, aspartic acid to asparagine, aspartic acid to aspartic acid, aspartic acid to glutamine. A conservative mutation can be glutamine to glutamine, glutamine to asparagine, glutamine to aspartic acid, glutamine to glutamine. A conservative mutation can be methionine to phenylalanine, methionine to isoleucine, methionine to valine, methionine to leucine, methionine to methionine. A conservative mutation can be tryptophan to tryptophan, tryptophan to phenylalanine, tryptophan to tyrosine.

Non-limiting examples of additional amino acid mutations can be: A to R, A to N, A to D, A to C, A to Q, A to E, A to G, A to H, A to I, A to L, A to K, A to M, A to F, A to P, A to S, A to T, A to W, A to Y, A to V, R to N, R to D, R to C, R to Q, R to E, R to G, R to H, R to I, R to L, R to K, R to M, R to F, R to P, R to S, R to T, R to W, R to Y, R to V, N to D, N to C, N to Q, N to E, N to G, N to H, N to I, N to L, N to K, N to M, N to F, N to P, N to S, N to T, N to W, N to Y, N to V, D to C, D to Q, D to E, D to G, D to H, D to I, D to L, D to K, D to M, D to F, D to P, D to S, D to T, D to W, D to Y, D to V, C to Q, C to E, C to G, C to H, C to I, C to L, C to K, C to M, C to F, C to P, C to S, C to T, C to W, C to Y, C to V, Q to E, Q to G, Q to H, Q to I, Q to L, Q to K, Q to M, Q to F, Q to P, Q to S, Q to T, Q to W, Q to Y, Q to V, E to G, E to H, E to I, E to L, E to K, E to M, E to F, E to P, E to S, E to T, E to W, E to Y, E to V, G to H, G to I, G to L, G to K, G to M, G to F, G to P, G to S, G to T, G to W, G to Y, G to V, H to I, H to L, H to K, H to M, H to F, H to P, H to S, H to T, H to W, H to Y, H to V, I to L, I to K, I to M, I to F, I to P, I to S, I to T, I to W, I to Y, I to V, L to K, L to M, L to F, L to P, L to S, L to T, L to W, L to Y, L to V, K to M, K to F, K to P, K to S, K to T, K to W, K to Y, K to V, M to F, M to P, M to S, M to T, M to W, M to Y, M to V, F to P, F to S, F to T, F to W, F to Y, F to V, P to S, P to T, P to W, P to Y, P to V, S to T, S to W, S to Y, S to V, T to W, T to Y, T to V, W to Y, W to V, Y to V, and any of the previously described mutations in reverse.

Any one of the aforementioned modifications, insertions, deletions, and/or mutations, can be made at any residue in an AAV sequence. The sequence may be a capsid sequence. In other cases, the sequence may not be a capsid sequence but rather a Rep and/or X sequence. The sequence may be in a VP1, VP2, and/or VP3 as previously described. In some cases, the sequence modification is of a loop of a capsid sequence, such as loop 3 and/or loop 4. In some cases, the modification is of a residue of a sequence in Table 27.

In some cases, a modification, such as insertion, deletion, and/or mutation is of a residue of a capsid polypeptide sequence in Table 27. In some cases, a modification is from 1-100, 100-200, 200-300, 300-400, 400-500, 500-600, 600-700, 700-800, or combinations thereof. In some cases, a modification is in a residue at position 200-300, 300-400, 400-500, 500-600 or combinations thereof. In some cases, a modification is in a residue at position 300-500 or combinations thereof. In an aspect, an insertion site is in the GH loop, or loop IV, of the AAV capsid protein, e.g., in a solvent-accessible portion of the GH loop, or loop IV, of the AAV capsid protein. For example, the insertion site is within amino acids 570-611 of AAV2, within amino acids 571-612 of AAV1, within amino acids 560-601 of AAV5, within amino acids 571 to 612 of AAV6, within amino acids 572 to 613 of AAV7, within amino acids 573 to 614 of AAV8, within amino acids 571 to 612 of AAV9, or within amino acids 573 to 614 of AAV10.

For example, the insertion site can be between amino acids 587 and 588 of AAV2, between amino acids 590 and 591 of AAV1, between amino acids 575 and 576 of AAV5, between amino acids 590 and 591 of AAV6, between amino acids 589 and 590 of AAV7, between amino acids 590 and 591 of AAV8, between amino acids 588 and 589 of AAV9, or between amino acids 589 and 590 of AAV10. In some cases, a modification is at position 452, 453, 466, 467, 468, 471, 585, 586, 587, and/or 588 of AAV2. In some cases, a modification is at position 452 or 453 of AAV2. In some cases, a modification is at position 587 or 588 of AAV2. In some cases, a modification is an insertion at position 452, 453, 466, 467, 468, 471, 585, 586, 587, and/or 588 of any one of SEQ ID NOs: 121-126. In some cases, a modification is an insertion at position 452, 453, 466, 467, 468, 471, 585, 586, 587, and/or 588 of SEQ ID NO: 121. In some cases, a modification is a mutation and the mutation is R585A or R588A of any one of SEQ ID NOs: 121-126. In some cases, a modification is a mutation and the mutation is R585A or R588A of SEQ ID NO: 121.

In some embodiments, a subject modified AAV capsid does not include any other amino acid modifications mutations, substitutions, insertions, or deletions, other than an insertion of from about 5 amino acids to about 11 amino acids in a loop (loop 3 and/or 4) relative to a corresponding unmodified AAV capsid protein. In other embodiments, a subject variant AAV capsid includes from 1 to about 25 amino acid insertions, deletions, or substitutions, compared to an unmodified AAV capsid protein, in addition to an insertion of from about 5 amino acids to about 11 amino acids in the loop 3 and/or loop 4 relative to an unmodified AAV capsid protein. In an embodiment, a subject AAV virion capsid does not include any other amino acid substitutions, insertions, or deletions, other than an insertion of from about 7 amino acids to about 10 amino acids in a GH loop or loop IV relative to a corresponding parental AAV capsid protein. In other embodiments, a subject AAV virion capsid includes from 1 to about 25 amino acid insertions, deletions, or substitutions, compared to the parental AAV capsid protein, in addition to an insertion of from about 7 amino acids to about 10 amino acids in the GH loop or loop IV relative to a corresponding parental AAV capsid protein. For example, in some embodiments, a subject AAV virion capsid includes from 1 to about 5, from about 5 to about 10, from about 10 to about 15, from about 15 to about 20, or from about 20 to about 25 amino acid insertions, deletions, or substitutions, compared to the parental AAV capsid protein, in addition to an insertion of from about 7 amino acids to about 10 amino acids in the GH loop or loop IV relative to a corresponding parental AAV capsid protein.

In some cases, a chimeric AAV capsid is provided herein. A chimeric capsid comprises a polypeptide sequence from at least 2 AAV serotypes. A chimeric capsid can comprise a mix of sequences selected from serotypes AAV1, AAV2, AAV3, AAV4, AAV5, AAV6, AAV7, AAV8, AAV9, AAV10, AAV11, and/or AAV12. In some cases, the chimeric serotypes are different between VP1, VP2, and/or VP3. In some cases, a chimeric capsid comprises sequences from at least 2 serotypes selected from: AAV4 and AAV6, AAV5 and AAV6, AAV11 and AAV6, AAV12 and AAV6, and any combination thereof. In some cases, a first AAV serotype can be AAV4 and a second serotype can be AAV6. In some cases, a first AAV serotype and a second AAV serotype of a chimeric AAV vector can be AAV11 and AAV6. In some cases, a first AAV serotype and a second AAV serotype of a chimeric AAV vector can be AAV12 and AAV6. In some cases, a chimeric capsid comprises sequences from: AAV2 and AAV5 or AAV2 and AAV6. In some cases, a chimeric capsid comprises sequences from: AAV2 and AAV5, AAV2 and AAV6, AAV2 and AAV8, AAV2 and AAV9, AAV2 and AAV1, and AAV2 and AAV12.

The modifications to an AAV provided herein can confer enhanced activity to the modified AAV as compared to an otherwise unmodified or wildtype AAV. Modifications provided herein can improve cell transduction, tropism, and/or reduce immunogenicity associated with the capsid.

In some cases, a modification provided herein enhances cellular transduction. Cellular transduction can refer to the ability of an AAV to infect a cell (in vivo or in vitro) and/or deliver a transgene into the cell.

In some cases, a modification provided herein enhances tropism. Enhanced tropism refers to gaining the ability to transduce cells through an extra receptor, as compared to an otherwise unmodified AAV. In some aspects, enhanced tropism can improve infectivity to an ocular cell, thereby improving gene therapy by utilization of the modified AAV. In some cases, a modification provided herein can improve tropism to an ocular cell selected from: bipolar, retinal ganglion, horizontal, amacrine, epithelial, retinal pigment, photoreceptor, or any combination thereof. In some cases, a modification improves tropism to a retinal cell.

Also provided herein are AAV vectors. AAV vectors comprise: inverted terminal repeats (ITRs), Rep, Cap, AAP, and X sequences. Typically, the AAV viral genome is flanked by the ITRs, which serve as packaging signal and origin of replication. The rep gene encodes a family of multifunctional proteins (Rep proteins) responsible for controlling viral transcription, replication, packaging, and integration in AAV integration site 1 (AAVS1). For AAV2, four Rep proteins are described. Expression of Rep78 and Rep68 is controlled by the AAV2-specific p5 promoter, while p19 controls expression of the smaller Rep proteins (Rep52 and Rep40). Rep68 and Rep40 are splice variants of Rep78 and Rep52, respectively. Numbers indicate the molecular weight. Expression of assembly-activating protein (AAP) and the viral capsid proteins VP1 (90 kDa), VP2 (72 kDa), and VP3 (60 kDa), all encoded in the cap gene, is controlled by the p40 promoter. The X gene is located at the 3′ end of the genome within a region shared with the cap gene and possesses its own promoter (p81). While the X protein seems to enhance viral replication, AAP is essential for capsid assembly. The three different VPs contribute in a 1 (VP1): 1 (VP2): 10 (VP3) ratio to the icosahedral AAV2 capsid.

A modified capsid protein disclosed herein can be isolated, e.g., purified. In some embodiments, a modified capsid disclosed herein is included in an AAV vector or an AAV virion (for example recombinant AAV virion rAAV). In other embodiments, such modified AAV vectors and/or AAV variant virions are used in an in vivo or ex vivo method of treating ocular disease in a primate retina, for example human retina.

Provided herein are also vectors that comprise modified AAV capsids. Any one of the previously described modifications can be encompassed in a vector provided herein. In some cases, an AAV vector comprises a modified capsid that comprises an exogenous sequence in at least two loops of a VP domain as compared to an otherwise comparable AAV capsid sequence that lacks the exogenous sequence. In some aspects, vectors provided herein can further comprise a transgene sequence.

### Pharmaceutical Composition

Described herein are pharmaceutical compositions comprising the plurality of viruses, plurality of viral particles, a cell comprising the plurality of viruses or plurality of viral particles described herein. In some embodiments, the pharmaceutical composition further comprises a pharmaceutically acceptable: carrier, excipient, or diluent. In some embodiments, the pharmaceutical composition comprises at least one additional active ingredient disclosed herein. In some embodiments, the pharmaceutical composition treats a disease or condition.

For in vivo delivery, the pharmaceutical compositions can generally be administered intravitreally or parenterally (e.g., administered via an intramuscular, subcutaneous, intratumoral, transdermal, intrathecal, etc., route of administration). In some embodiments, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, subretinally, suprachoroidally, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof to a subject in need thereof. In some aspects, a pharmaceutical composition can be used to treat a subject such as a human or mammal, in need thereof. In some cases, a subject can be diagnosed with a disease, e.g., ocular disease, vasculature disease, or cancer. In some aspects, pharmaceutical compositions are co-administered with secondary therapies.

Administrations can be repeated for any amount of time. In some aspects, administering is performed: twice daily, every other day, twice a week, bimonthly, trimonthly, once a month, every other month, semiannually, annually, or biannually.

Dosage treatment may be a single dose schedule or a multiple dose schedule. Moreover, the subject may be administered as many doses as appropriate. One of skilled in the art can readily determine an appropriate number of doses. In some aspects, a pharmaceutical composition is administered via intravitreal injection, subretinal injection, microinjection, or supraocular injection.

In practicing the methods of treatment or use provided herein, therapeutically effective amounts of the pharmaceutical composition described herein are administered to a mammal having a disease, disorder, or condition to be treated. In some embodiments, the mammal is a human. A therapeutically effective amount may vary widely depending on the severity of the disease, the age and relative health of the subject, the potency of the therapeutic agent used and other factors. The therapeutic agents, and in some cases, compositions described herein, may be used singly or in combination with one or more therapeutic agents as components of mixtures.

The pharmaceutical composition described herein may be administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical composition may be manufactured in a conventional manner, such as, by way of example only, by means of conventional mixing, dissolving, granulating, levigating, emulsifying, encapsulating, entrapping or compression processes.

In certain embodiments, the pharmaceutical composition provided herein includes one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, the pharmaceutical composition described herein is formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for rehydration into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms may be ensured by various antibacterial and antifungal agents, such as parabens, chlorobutanol, phenol, sorbic acid, and the like. In some cases, it is desirable to include isotonic agents, such as sugars, sodium chloride, and the like. Prolonged absorption of the injectable pharmaceutical form may be brought about by the use of agents delaying absorption, such as aluminum monostearate and gelatin.

In another aspect, dosage forms include microencapsulated formulations. In some embodiments, one or more other compatible materials are present in the microencapsulation material. Non-limiting example of materials includes pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Liquid formulation dosage forms for oral administration are optionally aqueous suspensions selected from the group including, but not limited to, pharmaceutically acceptable aqueous oral dispersions, emulsions, solutions, elixirs, gels, and syrups. In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical composition described herein is self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients.

Furthermore, the pharmaceutical composition optionally includes one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, the pharmaceutical composition optionally includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

### Kit

Disclosed herein, in some embodiments, are kits comprising the plurality of viruses, plurality of viral particles, a cell comprising the plurality of viruses or plurality of viral particles described herein. In some embodiments, the kit disclosed herein may be used to treat a disease or condition in a subject. In some embodiments, the kit comprises an assemblage of materials or components apart from the plurality of viruses.

In some embodiments, the kit described herein comprises components for selecting for a homogenous population of the plurality of viruses described herein. In some embodiments, the kit comprises the components for assaying the number of units of a biomolecule (e.g., the plurality of viruses) synthesized, and/or released or expressed on the surface by a host cell. In some embodiments, the kit comprises components for performing assays such as enzyme-linked immunosorbent assay (ELISA). The exact nature of the components configured in the kit depends on its intended purpose. For example, some embodiments are configured for the purpose of treating a disease or condition disclosed herein in a subject. In some embodiments, the kit is configured particularly for the purpose of treating mammalian subjects. In some embodiments, the kit is configured particularly for the purpose of treating human subjects.

Instructions for use may be included in the kit. In some embodiments, the kit comprises instructions for administration. Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the components may be in dissolved, dehydrated, or lyophilized form; they may be provided at room, refrigerated or frozen temperatures. The components are typically contained in suitable packaging material(s).

### Method of Manufacturing

Described herein, in some aspects, are methods for manufacturing the plurality of viruses or viral particles described herein. In some embodiments, the method comprises contacting a host cell with a sugar-based detergent or a formulation comprising the sugar-based detergent. In some embodiments, the method comprises releasing a plurality of viruses from a host cell, where the plurality of viruses are encoded by a vector in the host cell. In some embodiments, the method comprises contacting the host cell with a formulation comprising a sugar-based detergent for lysing the host cell, wherein said contacting with a sugar-based detergent releases the plurality of viruses from the host cell. In some embodiments, the contacting with the sugar-based detergent releases at least 10% %, at least 20%, at least 30%, at least 40%, at least 50%, at least 60%, at least 70%, at least 80%, at least 90%, or more of the plurality of viruses or viral particles encoded by a vector described herein from the host cell. In some embodiments, the plurality of viruses or viral particles comprises non-enveloped viruses. In some embodiments, the non-enveloped viruses comprise DNA viruses. Non-limiting example of the DNA virus can include, Adenoviruses, Parvoviruses, Polyomaviruses, or Anelloviruses. In some embodiments, the non-enveloped viruses comprise RNA viruses. Non-limiting example of the RNA virus can include Caliciviruses, Picornaviruses, Reoviruses, Astroviruses, or Hepeviridae. In some embodiments, the vector encodes at least one virus comprising at least one capsid. In some embodiments, the at least one capsid is an engineered capsid described herein. In some embodiments, the at least one capsid comprises a capsid of serotype AAV1, AAV2, AAV3, AAV4, AAV5, AAV6, AAV7, AAV8, AAV9, AAV10, AAV11, AAV12, AAV13, or a combination thereof.

In some embodiments, the contacting of the host cell with the formulation inactivates viruses that are different from the plurality of viruses encoded by the vector described herein. In some embodiments, the contacting of the host cell with the formulation inactivates viruses that are not encoded by the vector described herein. For example, Sf9 cells are often utilized as part of the viral manufacturing process, where inactivation of baculovirus is necessary. As such, the contacting of the host cell with the formulation described herein can release the plurality of viruses encoded by the vector, while also inactivates other viruses not encoded by the vector.

In some embodiments, the method comprises contacting with host cell with the sugar-based detergent or formulation comprising the sugar-based detergent. In some embodiments the method comprises contacting the host cell with a formulation comprising at least 0.0001%, at least 0.0005%, a least 0.001%, at least 0.005%, at least 0.01%, at least 0.05%, at least 0.1%, at least 0.5%, a least 1.0%, at least 5.0%, at least 10.0%, at least 20.0%, or at least 50.0% of the sugar-based detergent or APG described herein. In some embodiments, the method comprises supplement a medium for culturing the host cell described herein with the sugar-based detergent or a formulation comprising the sugar-based detergent, where the medium after the supplementation comprises at least 0.0001%, at least 0.0005%, a least 0.001%, at least 0.005%, at least 0.01%, at least 0.05%, at least 0.1%, at least 0.5%, a least 1.0%, at least 5.0%, at least 10.0%, at least 20.0%, or at least 50.0% of the sugar-based detergent or APG described herein.

In some embodiments, the contacting of the host cell with the sugar-based detergent, either directly or by supplementing with the medium for culturing the host cell, releases at least 10%, at least 20%, at least 30%, at least 40%, at least 50%, at least 60%, at least 70%, at least 80%, at least 90%, or more of the plurality of viruses or viral particles encoded by a vector described herein from the host cell. In some embodiments, the contacting of the host cell with the sugar-based detergent increases the yield of the plurality of viruses or viral particles released from the host cell compared to other methods not utilizing the sugar-based detergent. For example, the contacting of the host cell with the sugar-based detergent increases yield of the plurality of viruses or viral particles released from the host cell compared to yield of releasing the same plurality of viruses or viral particles from a comparable host cell by contacting the comparable host cell with a non-sugar-based detergent (e.g., Triton X-100). In some embodiments, the contacting of the host cell with the sugar-based detergent, either directly or by supplementing with the medium for culturing the host cell, increases yield of the plurality of viruses or viral particles released from the host cell by at least 0.1 fold, at least 0.2 fold, at least 0.4 fold, at least 0.8 fold, at least 1.0 fold, at least 1.2 fold, at least 1.5 fold, at least 2.0 fold, at least 3.0 fold, at least 4.0 fold, at least 5.0 fold, at least 10.0 fold, at least 20.0 fold, at least 50.0 fold, at least 100.0 fold, or more fold compared to yield of releasing the same plurality of viruses or viral particles from a comparable host cell by contacting the comparable host cell with a non-sugar-based detergent (e.g., Triton X-100).

In some embodiments, the contacting of the host cell with the sugar-based detergent, either directly or by supplementing with the medium for culturing the host cell, decreases aggregation of the plurality of viruses or viral particles released from the host cell compared to other methods not using the sugar-based detergent. For example, the contacting of the host cell with the sugar-based detergent decreases aggregation of the plurality of viruses or viral particles released from the host cell compared to aggregation the same plurality of viruses or viral particles released from a comparable host cell by contacting the comparable host cell with a non-sugar-based detergent (e.g., Triton X-100). In some embodiments, the contacting of the host cell with the sugar-based detergent, either directly or by supplementing with the medium for culturing the host cell, decreases aggregation of the plurality of viruses or viral particles released from the host cell by at least 0.1 fold, at least 0.2 fold, at least 0.4 fold, at least 0.8 fold, at least 1.0 fold, at least 1.2 fold, at least 1.5 fold, at least 2.0 fold, at least 3.0 fold, at least 4.0 fold, at least 5.0 fold, at least 10.0 fold, at least 20.0 fold, at least 50.0 fold, at least 100.0 fold, or more fold compared to aggregation of the same plurality of viruses or viral particles released from a comparable host cell by contacting the comparable host cell with a non-sugar-based detergent (e.g., Triton X-100).

In some embodiments, the contacting of the host cell with the sugar-based detergent, either directly or by supplementing with the medium for culturing the host cell, increases infectivity of the plurality of viruses or viral particles released from the host cell compared to other methods not utilizing the sugar-based detergent. For example, the contacting of the host cell with the sugar-based detergent increases infectivity of the plurality of viruses or viral particles released from the host cell compared to infectivity of the same plurality of viruses or viral particles released from a comparable host cell by contacting the comparable host cell with a non-sugar-based detergent (e.g., Triton X-100). In some embodiments, the contacting of the host cell with the sugar-based detergent, either directly or by supplementing with the medium for culturing the host cell, increases infectivity of the plurality of viruses or viral particles released from the host cell by at least 0.1 fold, at least 0.2 fold, at least 0.4 fold, at least 0.8 fold, at least 1.0 fold, at least 1.2 fold, at least 1.5 fold, at least 2.0 fold, at least 3.0 fold, at least 4.0 fold, at least 5.0 fold, at least 10.0 fold, at least 20.0 fold, at least 50.0 fold, at least 100.0 fold, or more fold compared to infectivity of the same plurality of viruses or viral particles released from a comparable host cell by contacting the comparable host cell with a non-sugar-based detergent (e.g., Triton X-100).

In some embodiments, the contacting of the host cell with the sugar-based detergent, either directly or by supplementing with the medium for culturing the host cell, decreases oxidative damage to the plurality of viruses or viral particles released from the host cell compared to other methods not utilizing the sugar-based detergent. For example, the contacting of the host cell with the sugar-based detergent decreases oxidative damage of the plurality of viruses or viral particles released from the host cell compared to oxidative damage of the same plurality of viruses or viral particles released from a comparable host cell by contacting the comparable host cell with a non-sugar-based detergent (e.g., Triton X-100). In some embodiments, the contacting of the host cell with the sugar-based detergent, either directly or by supplementing with the medium for culturing the host cell, decreases oxidative damage of the plurality of viruses or viral particles released from the host cell by at least 0.1 fold, at least 0.2 fold, at least 0.4 fold, at least 0.8 fold, at least 1.0 fold, at least 1.2 fold, at least 1.5 fold, at least 2.0 fold, at least 3.0 fold, at least 4.0 fold, at least 5.0 fold, at least 10.0 fold, at least 20.0 fold, at least 50.0 fold, at least 100.0 fold, or more fold compared to oxidative damage of the same plurality of viruses or viral particles released from a comparable host cell by contacting the comparable host cell with a non-sugar-based detergent (e.g., Triton X-100).

In some embodiments, the host cell is a bacteria cell. In some embodiments, the host cell is an eukaryotic cell. In some embodiments, the eukaryotic cell is a mammalian cell. In some embodiments, the eukaryotic cell is a yeast cell. In some embodiments, the eukaryotic cell is an insect cell (e.g., a Sf9 cell). In some embodiments, the host cell is a cell of a cell line. In some embodiments, the host cell is an immortalized cell. In some embodiments, the host cell can secrete the plurality of viruses or viral particles without being lysed. In such scenario, the sugar-based detergent or the formulation comprising the sugar-based detergent can be added to the medium for culturing the host cell, where the sugar-based detergent or the formulation comprising the sugar-based detergent can inactivate other viruses not encoded by the vector in the host cell in the medium. In some embodiments, the supplementation of the sugar-based detergent or the formulation comprising the sugar-based detergent to the medium can inactivate other viruses without the need to lower the pH of the medium. In some embodiments, the supplementation of the sugar-based detergent or the formulation comprising the sugar-based detergent to the medium can decrease aggregation of the plurality of viruses or viral particles encoded by a vector in the host cell.

In some embodiments, the method described herein comprises releasing a plurality of viruses encoded by a vector in the host cell, the method comprising contacting the host cell with a sugar-based detergent for lysing the host cell, wherein said contacting with the a sugar-based detergent releases the plurality of viruses that are at least 50% more infective compared to an infectivity of a second plurality of comparable viruses that are obtained by releasing the second plurality of the comparable viruses by a method that does not use the sugar-based detergent. In some embodiments, the plurality of viruses, upon released by contacting with the sugar-based detergent, is at least 60%, at least 70%, at least 80%, at least 90%, or more infective compared to the infectivity of the second plurality of comparable viruses. In some embodiments, the infectivity is determined by a viral titer assay. In some embodiments, the second plurality of comparable viruses, after being released from a comparable hose cell, forms at least 10% more aggregate compared by the plurality of viruses.

In some embodiments, the method described herein comprises at least one of following for isolating the plurality of viruses from the host cell: filtration, chromatography, centrifugation, buffer exchange, or sterilization. In some embodiments, the at least one or a combination of filtration, chromatography, centrifugation, buffer exchange, or sterilization can further process the plurality of the viruses or viral particles described herein for treatment of a disease or condition or for formulation into a pharmaceutical composition. In some embodiments, the method utilizing the sugar-based detergent or formulation comprising the sugar-based detergent can substitute at least one or a combination of filtration, chromatography, centrifugation, buffer exchange, or sterilization. For example, the contacting of the host cell or the medium for culturing the host cell with the sugar-based detergent or formulation comprising the sugar-based detergent can substitute the sterilization, where the sugar-based detergent or formulation comprising the sugar-based detergent inactivates other viruses not suitable for treatment of disease or condition or not suitable to be formulated into a pharmaceutical composition.

### Method of Treatment

Provided herein are methods of treating a disease or condition. A method of treatment can comprise introducing to a subject in need the plurality of viruses, plurality of viral particles, a cell comprising the plurality of viruses or plurality of viral particles, a pharmaceutical composition, or a combination thereof. In some embodiments, administration is by any suitable mode of administration, including systemic administration (e.g., intravenous, intravitreal, subretinal, or etc.). In some embodiments, the subject is human.

In some embodiments, the method comprises treating a disease or condition in a subject in need thereof by administering to the subject a therapeutically effective amount of the plurality of viruses, plurality of viral particles, a cell comprising the plurality of viruses or plurality of viral particles, a pharmaceutical composition, or a combination thereof. In some embodiments, the method treats a disease or condition, wherein once of the administering of the plurality of viruses, plurality of viral particles, a cell comprising the plurality of viruses or plurality of viral particles, a pharmaceutical composition, or a combination thereof described herein is curative of the disease or condition. In some embodiments, the method treats a disease or condition, wherein the plurality of viruses, plurality of viral particles, a cell comprising the plurality of viruses or plurality of viral particles, a pharmaceutical composition, or a combination thereof does not comprise daily administration.

In some embodiments, the plurality of viruses, plurality of viral particles, a cell comprising the plurality of viruses or plurality of viral particles, a pharmaceutical composition, or a combination thereof is administered at least once during a period of time (e.g., every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year). In some embodiments, the plurality of viruses, plurality of viral particles, a cell comprising the plurality of viruses or plurality of viral particles, a pharmaceutical composition, or a combination thereof is administered two or more times (e.g., 2, 3, 4, 5, 6, 7, 8, 9, 10, 11, 12, 13, 14, 15, 16, 17, 18, 19, 20, 30, 40, 50, 60, 70, 80, 90, 100 times) during a period of time.

In some embodiments, the method comprises administering the plurality of viruses, plurality of viral particles, a cell comprising the plurality of viruses or plurality of viral particles, a pharmaceutical composition, or a combination thereof in a therapeutically-effective amount by various forms and routes including, for example, oral, or topical administration. In some embodiments, the plurality of viruses, plurality of viral particles, a cell comprising the plurality of viruses or plurality of viral particles, a pharmaceutical composition, or a combination thereof may be administered by intravitreal, subretinal, suprachoroidal, parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, subarachnoid, or intraspinal administration, e.g., injection or infusion. In some embodiments, the plurality of viruses, plurality of viral particles, a cell comprising the plurality of viruses or plurality of viral particles, a pharmaceutical composition, or a combination thereof may be administered by absorption through epithelial or mucocutaneous linings (e.g., oral mucosa, rectal and intestinal mucosa administration). In some embodiments, the plurality of viruses, plurality of viral particles, a cell comprising the plurality of viruses or plurality of viral particles, a pharmaceutical composition, or a combination thereof is delivered via multiple administration routes.

In some embodiments, the method comprises administering the plurality of viruses, plurality of viral particles, a cell comprising the plurality of viruses or plurality of viral particles, a pharmaceutical composition, or a combination thereof by intravenous infusion. In some embodiments, the plurality of viruses, plurality of viral particles, a cell comprising the plurality of viruses or plurality of viral particles, a pharmaceutical composition, or a combination thereof is administered by slow continuous infusion over a long period, such as more than 24 hours. In some embodiments, the plurality of viruses, plurality of viral particles, a cell comprising the plurality of viruses or plurality of viral particles, a pharmaceutical composition, or a combination thereof is administered as an intravenous injection or a short infusion. In some embodiments, the plurality of viruses, plurality of viral particles, a cell comprising the plurality of viruses or plurality of viral particles, a pharmaceutical composition, or a combination thereof is administered via vitreous route. In some embodiments, the plurality of viruses, plurality of viral particles, a cell comprising the plurality of viruses or plurality of viral particles, a pharmaceutical composition, or a combination thereof may be administered in a local manner, for example, via injection of the agent directly into an organ, optionally in a depot or sustained release formulation or implant.

In some embodiments, the plurality of viruses, plurality of viral particles, a cell comprising the plurality of viruses or plurality of viral particles, a pharmaceutical composition, or a combination thereof may be administered in conjunction with other therapies, for example, an antiviral therapy, a chemotherapy, an antibiotic, a cell therapy, a cytokine therapy, or an anti-inflammatory agent. In some embodiments, the plurality of viruses, plurality of viral particles, a cell comprising the plurality of viruses or plurality of viral particles, a pharmaceutical composition, or a combination thereof may be administered before, during, or after the occurrence of a disease or condition, and the timing of administering the composition containing a therapeutic agent may vary. In some cases, the plurality of viruses, plurality of viral particles, a cell comprising the plurality of viruses or plurality of viral particles, a pharmaceutical composition, or a combination thereof may be used as a prophylactic and may be administered continuously to subjects (e.g., the subject for immunization or the subject for treatment). Prophylactic administration may lessen a likelihood of the occurrence of the infection, disease or condition, or may reduce the severity of the infection, disease or condition.

The plurality of viruses, plurality of viral particles, a cell comprising the plurality of viruses or plurality of viral particles, a pharmaceutical composition, or a combination thereof may be administered to a subject before the onset of the symptoms. The plurality of viruses, plurality of viral particles, a cell comprising the plurality of viruses or plurality of viral particles, a pharmaceutical composition, or a combination thereof may be administered to a subject (e.g., the subject for immunization or the subject for treatment) after (e.g., as soon as possible after) a test result.

In some cases, an administration of the plurality of viruses, plurality of viral particles, a cell comprising the plurality of viruses or plurality of viral particles, a pharmaceutical composition, or a combination thereof is sufficient to reduce at least a symptom of a disease or condition, treat the disease or condition, and/or eliminate the disease or condition. In some cases, improvements of diseases or conditions can be ascertained by any of the provided diagnostic assays. In other cases, an improvement can be obtained via an interview with the treated subject. For example, a subject may be able to communicate to an attending physician that their vision is improved as compared to their vision prior to administration of a subject pharmaceutical. In other cases, an in vivo animal model may be used to ascertain reduction of a disease or condition after treatment. Suitable animal models include mouse models, primate models, rat models, canine models, and the like.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following illustrative examples are representative of embodiments of the stimulation, systems, and methods described herein and are not meant to be limiting in any way.

### Example 1. Virus Manufacturing

AAV viral particle (e.g., AAV virus or AAV vector such as AAV2.N54-Aflibercept, AVMX-110) in a bioreactor after completion of fermentation can be harvested, purified, and formulated into a therapeutic described herein. An exemplary process flow is outlined in FIG. 1. During the entire process of AAV downstream manufacturing, samples can be taken for various types of tests as shown in FIG. 1. During the manufacturing process, samples will be taken at various points of process steps for various purposes of testing in order to achieve accurate control of processes, and therefore to maintain the process parameters reliably controlled and consistent manufacture quality drug substances (DS such as a therapeutic described herein).

### Sf9 Cell Lysis and Virus Inactivation

This step is the first unit operation of AAV viral particle downstream recovery process. When the cell culture reached its 20-30% viability after growth peak, 20× lysis buffer and 25-50 IU/mL of Benzonase™ can be directly added into the bioreactor, adjusting pH to 7.2-7.5 with 2 M Tris base. The 20× lysis buffer is composed of 500 mM Tris, pH 7.5, 40% (v/v) APG, 4% (w/v) SLS, 100 mM MgCl2. Benzonase™ is a product recombinant nuclease from MilliporeSigmaMerck, with enzymatic activity is >250,000 IU/mL. The bioreactor is stirred at 60-190 rpm further at 37° C. for 90-120 minutes and the bioreactor pH is maintained at 7.4±0.20.

### Material and Buffer Preparation

The raw materials used in the buffer preparation should be GMP grade meeting with quality assurance certified release criteria. The final buffer can be added to the bioreactor after verification of cell culture status and after sample taken for analysis of rAAV distribution inside cells or culture supernatant.

### Viral Inactivation

During the lysis of Sf9 cell culture by microfluidics in the process, there are two orthogonal virus inactivation mechanisms achieved: inactivation by the nonionic detergent, APG, used for dissociation of AAV particles from the cell lysate; and inactivation by the ionic detergent like Sarcosyl. The purification process can be started from the virus inactivation with APG or Sarcosyl at 1.25±0.1% (w/v). Table 1 illustrates exemplary Sf9 cell Rhabdovirus inactivation by a single pass of a microfluidics device (MFD) (pfu/mL) (log 10). Table 2 illustrates exemplary AAV inactivation by a single pass of MFD (pfu/mL) (log 10).

TABLE 1
Sf9 cell Rhabdovirus inactivation by
a single pass of MFD (pfu/mL) (log10)

| | No | | | | Claimed |
|---|---|---|---|---|---|
| Pre | treatment | 10,000 psi | 15,000 psi | 20,000 psi | LRF |
| PC | 7.0 | | | | |
| NC | ≤0.5 | ≤0.5 | ≤0.5 | ≤0.5 | ≥6.5 |
| Unprocessed | 7 | ≤0.5 | ≤0.5 | ≤0.5 | ≥6.5 |
| bulk | | | | | |
| LAVA | 8.0 | | | | ≥7.5 |

TABLE 2
AAV by a single pass of MFD (pfu/mL) (log10)

| | No | | | | Claimed |
|---|---|---|---|---|---|
| Pre | treatment | 10,000 psi | 15,000 psi | 20,000 psi | LRF |
| PC | 7.0 | | | | |
| NC | ≤0.5 | ≤0.5 | ≤0.5 | ≤0.5 | ≥6.5 |
| Unprocessed | 7 | ≤0.5 | ≤0.5 | ≤0.5 | ≥6.5 |
| bulk | | | | | |
| LAVA | 8.0 | | | | ≥7.5 |

### Depth Filtration

The lysate can be filtered through a prefilter, a microfiltration filter, a 5.0 μm filter in tandem with a depth filter of 3-layer of PES membrane (Millipore, 1.2/0.5/0.2 μm filters (PES) sequentially using Millipore SHC capsule, at 1-10 L/min (choose a proper surface area of filters. The filtrate is called clarified cell lysate (CCL). This type of design can achieve good filtration with 1.2/0.5 μm pore size, and the 0.22 μm is a layer for sterilization to remove microbial contamination, minimize bioburden and endotoxin level. The 1.2/0.5/0.2 μm filter should be gamma irradiated and secure in its integral package. The buffer for membrane flushing can include: 20 mM Tris-HCl, pH 8.0±0.2; 100 mM Sodium Chloride (NaCl); and 1 mM EDTA. The depth filtrate is called clarified Sf9 cell lysate (CCL). CCL is processed immediately via tangential flow filtration (TFF) through a 300 kDa polyester sulfone (PES) membrane (Millipore, MA). It can be stored at 2-8° C. for TFF. The purpose of the TFF step is to achieve a partial purification. Theoretically the molecules under 300 kDa can be filtrate through the membrane while those larger than 300 kDa can be retained in the retentate. The virally inactivated CCL (1× vol) can be filtered through a 300 kDa TFF filter (PES) and diluted with 5 vol of 20 mM Tris, 100 mM NaCl, 1 mM EDTA (TNE buffer) and continued to concentrate by TFF and repeat the process one more cycle. The final retentate is used as the load of affinity chromatographic step.

### Capturing of Viral Particle by Affinity Column Chromatography

The TFF retentate can be further processed through an affinity column chromatography using POROS™ CaptureSelect™ AAVx resin (Thermo Fisher). POROS™ CaptureSelect™ AAVx resin comprises binding capacity of at least or at most 1×10¹⁴ vg/mL. The column dimension is about 25×10 cm (column volume 2 L). The column height is 10±1.0 cm. Estimate binding capacity per column can be 2×10¹⁷ vg per cycle. Load can be about 200 L filtrate. Table 3 illustrates exemplary buffer used for column chromatography.

TABLE 3
Exemplary buffer used for column chromatography

| | | | Conduc- | |
|---|---|---|---|---|
| Name | Composition | PH | tivity | Volume |
| Equilibration | 20 mM Tris | 7.6 ± 0.2 | measure | 3 CV |
| buffer | 150 mM NaCl | | | |
| | 1 mM EDTA | | | |
| | 0.005% (w/v) | | | |
| | Poloxamer 188 | | | |
| Wash 1 buffer | 20 mM Tris | 9.0 ± 0.2 | measure | 3 CV |
| | 1000 mM NaCl | | | |
| | 1 mM EDTA | | | |
| | 0.005% (w/v) | | | |
| | Poloxamer 188 | | | |
| Wash 2 | 25 mM Tris | 5.0 ± 0.2 | measure | 2 CV |
| | 1 mM EDTA | | | |
| | 0.005% (w/v) | | | |
| | Poloxamer 188 | | | |
| Elution | 200 mM glycine | pH 2.5 + 0.2 | measure | 3-5 CV |
| | 500 mM Arginine | | | |
| | 200 mM NaCl | | | |
| | 0.005% (w/v) | | | |
| | Poloxamer 188 | | | |
| Strip solution | 150-200 mM | | measure | 3 CV |
| | phosphoric acid | | | |
| Regeneration | 6M Guanidine HCl | pH 7.0 ± 0.2 | measure | 2 CV |
| buffer | | | | |
| Storage buffer | 20% ethanol | pH 7.0 ± 0.2 | measure | 2 CV |

### Anion Exchange Column Chromatography

Enrichment of full capsid can be performed using POROS™ XQ column. This is binding and elution mode in that the viral particle can bind onto POROS™ XQ resin and can be eluted by a step gradient formed with sodium chloride (NaCl), after thorough wash. The AAVx affinity column eluate can be diluted, conditioned for the pH and conductivity to appropriate values suitable for AEX resin. The resultant eluate can be designated. Binding capacity is about 1-5×10¹³ vg/mL resin. Column dimension is about 18×20 cm (20.3 L resin) and expected binding capacity is 2×10¹⁷ vg per batch. Loading is about 2×10¹⁷ vg in 100 L.

### Example 2. Viral Clearance by AAVx Affinity Chromatography

The BAC-to-AAV technology has been utilized to produce rAAV. It is not known if the purified fractions of recombinant AAVs produced with the baculovirus system contain infectious recombinant baculovirus (rBV) particles.

In this study, a small quantity of Sf9-V432AG cells in suspension culture from a 3 L bioreactor was harvested, mechanically disrupted without adding detergents or Benzonase to eliminate the effect of rBV inactivation by detergent or Benzonase, and clarified by the depth filtration (DF) and filtered via a 0.2 μm filter. rBV-GFP stock 20-VS-BV-01 was spiked into the filtrate at 2% (v/v) immediately before loading the sample on an AAVx affinity column. The AAVx column chromatography was processed as a mock AAV purification procedure and the eluate was collected, neutralized to pH 7.5, and 0.2 μm filtered. The previously developed fluorescent TCID50 (F-TCID50) assay was applied to evaluate the baculovirus clearance by AAVx affinity column purification. Table 4 illustrates the materials and methods used for this study.

TABLE 4
Materials and methods for the affinity
chromatography study in Example 2

| Product name | Vendor | Cat No. |
|---|---|---|
| Sf-9 | Expression Systems | 94-006F |
| rBV-V445 (GFP) | In-house | 20-VS-BV-01 |
| ESF AF medium | Expression Systems | 99-300-01 |
| 96-well plate | Corning | 3340 |
| Multi-Channel pipettor | Eppendorf | 1200 |
| BSC | Labconco | 362090437 |
| 28° C. incubator | Forma Scientific | 3326 |
| microscope | Amscope | IN300TC-FL |
| Countless cell counting | Invitrogen | C10283 |
| chamber slides | | |
| Countess II FL | Life Technologies | A27974 |
| −80 freezer | Thermo Electron Corp. | 8606 |
| 3 L autoclavable bioreactor | Applikon | ADI |
| | Biotechnology | Z5100002M0 |
| Bio Console | Applikon | ADI 1025 |
| | Biotechnology | |
| Bio Controller | Applikon | ADI 1010 |
| | Biotechnology | |
| Depth filtration filter | Millipore Sigma | C0HC23CL3 |
| Filtration pump | Cole-Parmer | Easy-load II |
| | Masterflex L/S | |
| POROS ™ CaptureSelect ™ | Thermo Fisher | A36741 |
| AAVx Affinity resin | | |
| Sterile filtration filter | Millipore Sigma | KHGES006FF3 |
| AKTA Explorer | GE Healthcare | NA |
| POROS ™ CaptureSelect ™ | Thermo Scientific | A36741 |
| AAV Resins | | |
| NaCl, USP | Sigma Aldrich | S7653-1KG |
| Na₂HPO₄, USP | Sigma Aldrich | 30435-500G |
| NaCl, USP | Sigma Aldrich | S7653-1KG |
| Na₂HPO₄, USP | Sigma Aldrich | 30435-500G |
| NaH₂PO₄, USP | Sigma Aldrich | 04269-1KG |
| Tris base, USP | Gold Bio | E-210-1 |
| Glycine | Avantor Performance | 4059-02 |
| HCl, USP | Ward's Science | 7647-01-0 |
| EDTA | Gold Bio | E-210-1 |
| Arginine | Amresco | 0877-500G |
| Guanidine | Thermo Scientific | J65661-36 |
| Benzyl alcohol | Sigma Aldrich | 305197-1L |
| Sterile MilliQ water/WFI | CalBiochem | 4.86505.9020 |

### V432AG Cell Culture Growth in a 3 L Applikon Bioreactor

One liter (1 L) of V432AG cell culture was inoculated at 1×10⁶ cells/mL into a 3 L Applikon bioreactor vessel with temperature at 28° C., stirring speed at 180 rpm, and oxygen level at 21% using a porous sparger. The cell culture was monitored daily by measuring total cell counts and viable cells using a cell counter.

### Cell Lysis, Depth/Sterile Filtration, and rBV Spiking

One liter of cell culture was sonicated and mechanically lysed. The cell lysate was clarified by depth filtration using a MilliporeSigma depth filter, Millistak+ media in uPod format C0HC (MilliporeSigma: cat #MC0HC23CL3, Lot #CP0KB15741). The filter surface area is 23 cm². For the 1 L culture, three filter devices were used to clarify the entire lysate. The three filter devices were connected to the lysate container, in parallel, via inlets using Masterflex 14 sized tubing which passed through the pump head of a Cole-Pormer Masterflex L/S, easy Load II. The outlets of the three depth filters were connected to the inlet of a 0.2 μm pore-sized sterile filter unit, Opticap XL 600 Capsule, Express SHC 0.5/0.2 μm having a surface area of 590 cm² (MilliporeSigma: cat #KHGES006FF3, Lot C4HA75555). Filtration flow rate was maintained at a constant 3 mL/min and the filters were flushed with 500 mL PBS to chase retented lysate. Ten (10) mL of rBV-GFP stock 20-VS-BV-01, after, optionally, being treated with DNaseI at 37° C. for one hour, was spiked into 500 mL of filtrate (2% (v/v)) after DF and 0.2 μm filtration. An aliquot of the rBV-spiked sample (sample Load) was saved for the F-TCID50 assay.

### Affinity Column Chromatography

The DF/0.2 μm filtrate was processed through an affinity column chromatography using POROS™ CaptureSelect™ AAVx resin (Thermo Fisher, A36741) as a mock AAV downstream purification. About ten (10) mL (50:1) of the AAVx column eluate was collected and immediately neutralized to pH 7.5 using 1 M Tris.HCl pH 10. An aliquot of the neutralized eluate was 0.2 μm filtered (sample Eluate) and analyzed by the F-TCID₅₀ assay.

### Toxicity of AAVx Elution Buffer (pH7.5) on Sf-9 Cells

Sf-9 cells were seeded in two 96-well plates at 4×10⁵ cells/mL, 50 μL/well, and incubated at 28° C. for more than 30 min for cells to settle. AAVx elution buffer (pH7.5) was 1:3.2 serially diluted with ESF AF medium. ESF AF medium was added to the last 96-well column as the negative control. Each diluent, from low to high, was added at 50 L/well to each well of a column, from column #11 to column #1 of the 96-well plate. Finally, ESF AF medium containing 20-VS-BV-01 stock at 1:300,000 dilution, was added to four wells of each 96-well column at 50 μL/well. Cells were examined by visible/florescent microscopy after incubating at 28° C. for 6-8 days.

Interference with rBV Infectivity by AAVx Elution Buffer (pH7.5)

Sf-9 cells were seeded in four 96-well plates at 4×10⁵ cells/mL, 50 μL/well, and incubated at 28° C. for more than 30 min for cells to settle. Ten (10) mL of each AAVx elution buffer (pH7.5) diluent was prepared at 1:3.2, 1:3.22 (1:10), and 1:3.23 (1:32) with ESF AF medium. rBV-GFP stock 20-VS-BV-01 (10 μL) was spiked into 990 μL of ESF AF medium and each of the above diluents. The F-TCID50 assay was performed using ESF AF medium and each diluent using 1:3.2 serial dilutions.

### Evaluation of rBV Clearance by AAVx Column Chromatography

Sf-9 cells were seeded in 96-well plates at density of 4×10⁵ cells/well, 50 μL/well, and incubated at 28° C. for more than 30 min for the cells to settle. Virus-spiked sample Load and sample Eluate, 100 μL of each, were added to a sterile 15-mL tube containing 4900 μL (1:50) of ESF AF medium and vortexed to completely mix.

### Toxicity of AAVx Elution Buffer (pH7.5) on Sf-9 Cell Cultures

Baculovirus assay indicator cells, Sf-9 cells, were seeded in 96-well cell culture plates. Serially diluted AAVx elution buffer with pH adjusted to 7.5 was added to the cell culture wells and incubated at 28° C. for 6-8 days prior to observation and scoring under a visible/Fluorescent microscope. The results showed that no significant morphological changes of the cells were observed at any dilutions tested compared to the controls. Consistently, GFP signals were observed in all the wells with rBV-GFP. Therefore, 1:3.2 dilution of AAVx elution buffer at pH7.5 was non-cytotoxic to Sf9 cells.

Interference with rBV Infectivity by AAVx Elution Buffer (pH7.5)

In addition to cytotoxicity, samples that showed no signs of cytotoxicity may exhibit viral interference by interfering with the ability of the virus to establish infection in the host cells. The infectivity titers of rBV-GFP (20-VS-BV-01) in AAVx elution buffer diluents prepared with ESF AF medium at 1:3.2, 1:3.2² (1:10), and 1:3.2³ (1:32), respectively, were compared to the rBV's infectivity titer in ESF AF medium. The results indicated that there was no interference at the dilutions of 1:3.2² (1:10) and above. Therefore, 1:3.2² (1:10) dilution of AAVx elution buffer was regarded as a non-interfering dilution. At the dilution of 1:3.2, rBV infectivity was reduced 1.5±0.4 log₁₀ (Table 5), indicating 1:3.2 dilution of AAVx elution buffer at pH7.5 was regarded as the interfering dilution.

TABLE 5
rBV interference by AAVx elution buffer pH 7.5

| Dilution | Log₁₀ (TCID₅₀)/mL | LRV |
|---|---|---|
| ESF AF (PC*) | 6.1 ± 0.2 | — |
| AAVx eluate 1:3.2 | 4.6 ± 0.3 | 1.5 ± 0.4 |
| AAVx eluate 1:3.2² | 6.0 ± 0.3 | 0.1 ± 0.4 |
| AAVx eluate 1:3.2³ | 6.2 ± 0.3 | 0.1 ± 0.4 |

*PC—positive control

### Assessment of rBV Clearance by AAVx Affinity Chromatography

V432AG Sf-9 cells from bioreactors was mechanically lysed without detergents/Benzonase prior to depth filtration and 0.2 μm filtration. rBV-GFP stock 20-VS-BV-01 was spiked in the processed samples at 2% (v/v) immediately before loading onto an AAVx column, used as a mock AAV purification process. The AAVx column eluate was collected, immediately neutralized to pH7.5, and 0.2 μm filtered. The Sample Eluate was diluted 1:50 with ESF AF medium. since 500 mL of the Sample Load was reduced to 10 mL eluate volume. AAVx eluate at pH7.5 was non-cytotoxic and non-interfering at this dilution based on the toxicity and interference studies. The results of F-TCID50 assay showed that the log₁₀ reduction value (LRV) was more than 4.8±0.3 by the AAVx affinity chromatography (Table 6).

TABLE 6
rBV infectious titers before and after AAVx
affinity chromatography purification

| Sample | Log₁₀(TCID₅₀)/mL |
|---|---|
| AAVx Load | 6.1 ± 0.3 |
| AAVx Eluate | ≤1.3 |
| LRV | ≥4.8 ± 0.3 |

AAVx elution buffer was adjusted to pH 7.5 and used as an alternative to a mock AAVx eluate pH 7.5 without rBV spiking for the toxicity and interference studies of AAVx eluate pH7.5. AAVx elution buffer pH 7.5 was non-cytotoxic at 1:3.2 dilution and above, and non-interfering at 1:3.22 (1:10) dilution and above. The results were informative for a research purpose. Real mock AAVx eluate pH 7.5 without rBV spiking will be used for toxicity and interference assays in the future GMP/GLP manufacturing processes.

Toxicity and interference assays for the AAVx load were not performed in this study because no chemicals or reagents were added to the V432AG cell culture. Toxicity and interference by a mock AAVx load will be performed during future GMP/GLP studies.

The LRV of rBV clearance was ≥4.8±0.3 by AAVx affinity column chromatography during the mock AAV purification. However, the 1:50 dilution of the sample eluate might not be necessary since AAVx eluate buffer (sample eluate) was non-cytotoxic and non-interfering at the 1:10 dilution. Non-diluted sample eluate will be used during future GMP/GLP studies and total TCID50 or AAVx Load and AAVx Eluate will be calculated respectively. The difference of the volume size of AAVx Load and AAVx Eluate will contribute to a bigger LRV of rBV clearance at the AAVx affinity column chromatography step.

### Example 3. RBV Clearance by Viresolve NFR OptiScale-25 Capsule (NPR Filter)

The BAC-to-AAV technology has been utilized to produce rAAV. This study was designed to determine if the purified fractions of recombinant AAVs produced with the baculovirus system contain infectious recombinant baculovirus (rBV) particles. In this study, an AAV full band was collected after ultracentrifugation in the presence of 1.34 g/mL CsCl. A small amount of the AAV full band was diluted with AVMX Formulation Buffer 1 and the rBV-GFP stock 20-VS-BV-01 was spiked in the diluent at 2% (v/v) immediately before loading the sample on an NFR filter for viral filtration. Six fractions of the filtrate were collected and AAV and rBV titers in each of the fractions were quantified by qPCR analysis to evaluate AAV recovery and rBV removal by NFR. In addition, the infectivity of the rBV before and after NFR filtration was measured using a previously developed fluorescent TCID₅₀ (F-TCID50) assay and compared with the qPCR results. Table 7 illustrates the materials and methods for Example 3.

TABLE 7
Materials and methods

| Product name | Vendor | Cat No. |
|---|---|---|
| Sf-9 | Expression Systems | 94-006F |
| rBV-V445 (GFP) | In-house | 20-VS-BV-01 |
| AAV2.N54-AMI120 | In-house | 21-026 |
| AAVx eluate, pH 7.5 | In-house | 21-079 |
| ESF AF | Expression Systems | 99-300-01 |
| 96-well plate | Corning | 3340 |
| Multi-Channel pipettor | Eppendorf | 1200 |
| BSC | Labconco | 362090437 |
| 28° C. incubator | Forma Scientific | 3326 |
| microscope | Amscope | IN300TC-FL |
| Countless cell counting | Invitrogen | C10283 |
| chamber slides | | |
| Countess II FL | Life technologies | A27974 |
| −80 freezer | Thermo Electron | 8606 |
| | Corp. | |
| Filtration pump | Cole-Pormer | Easy-load II |
| | Masterflex L/S | |
| PowerUp SYBR Green | Thermo Fisher | 100029284 |
| Master Mix | | |
| Sterile filtration filter | Millipore Sigma | KHGES006FF3 |
| Viresolve NFR OptiScale- | Millipore Sigma | SZRV025NB9 |
| 25 Capsule (NFR filter) | on site | |
| Ultracentrifuge Tube | Seton | 3041 |
| CsCl | Fisher Scientific | BP1595-1 |
| CsCl at 1.34 g/cc in PBS | In house | 07272021 |
| 70 Ti centrifuge rotor | Beckman | 96U3721 |
| AVMX2 formulation buffer 1 | In house | Freshly prepared |
| QIAamp Viral RNA Mini Kit | Qiagen | 52906 |
| PowerUp SYBR Green | Thermo Fisher | 100029284 |
| Master Mix | | |

Toxicity of 1.34 g/mL CsCl and AVMX Formulation Buffer 1 on Sf-9 Cell Cultures

Sf-9 cells were seeded in two 96-well plates at 4×10⁵ cells/mL, 50 μL/well, and incubated at 28° C. for more than 30 min to enable cells to settle. CsCl titrations, starting at 1.34 g/mL and serially diluted 1:3.2 with ESF AF medium, were added to each well of the first plate at 50 μL/well, with the same dilution on each plate column. ESF AF medium was added to the last column as the negative control. Similarly, AVMX Formulation Buffer 1 was serially diluted at 1:3.2 and added to the other plate seeded with Sf9 cells. Finally, medium containing 21-VS-BV-01 stock at 1:100,000 dilution was added to the bottom four wells of each plate column. Cells were examined by visible/florescent microscopy after incubation at 28° C. for 6-8 days.

Interference with rBV Infectivity by 1.34 g/mL CsCl or AVMX2 Formulation Buffer 1

Sf-9 cells were seeded in four 96-well plates at 4×10⁵ cells/mL, 50 μL/well, and incubated at 28° C. for more than 30 min to enable cells to settle. Ten (10) milliliters of each 1.34 g/mL CsCl diluent at 1:3.24 (1:100), 1:3.25 (1:320), and 1:3.26 (1:1000) with ESF AF medium were prepared. Similarly, ten (10) milliliters of each AVMX Formulation Buffer 1 diluent at 1:3.2, 1:3.22 (1:10), and 1:3.23 (1:32) with ESF AF medium were prepared. Ten microliter (10 μL) of rBV-GFP stock 20-VS-BV-01 was spiked in 990 μL of ESF AF medium or each above diluent at 1% (v/v), respectively. The F-TCID50 assay was performed with medium and each corresponding diluent.

### rBV Clearance by NFR Filtration

After ultracentrifugation, 1.5 mL out of 5.6 mL of AAV full band was diluted with AVMX Formulation Buffer to the final volume of 40 mL and the AAV titer was determined at ˜2.4×1012 vg/m by qPCR analysis. rBV-GFP stock 20-VS-BV-01 was spiked into the AAV solution at 2% (v/v) immediately before loading onto an NFR filtration system for AAV recovery and rBV clearance analyses. One milliliter (1 mL) of rBV-spiked sample was saved and filtered by a 0.22-μm filter for qPCR and F-TCID50 assays before viral filtration, and the remaining sample was processed for NFR filtration by a Millipore team onsite. Six fractions of filtered samples were collected after the filtration and the process was recorded. The titers of AAV and rBV of each fraction were evaluated by qPCR assays and the rBV infectivity of each fraction was evaluated by F-TCID50 assay using 1:3.2 serial dilutions.

Toxicity on Sf-9 Cells and Interference with rBV Infectivity by 1.34 g/mL CsCl

Baculovirus assay indicator, Sf-9 cells, were seeded in 96-well cell culture plates. Serially diluted with medium, 1.34 g/mL CsCl, was added to the cell culture wells and incubated at 28° C. for 6-8 days prior to observation and scoring under a visible/Fluorescent microscope (Amscope, IN300TC-FL). The results showed that significant morphological changes of the cells were observed in the wells with dilutions at 1:3.23 (1:32) and lower. Consistently, GFP signals were observed only in the wells with dilutions at 1:3.24 (1:100) and above in the presence of rBV-GFP (data not shown). Therefore, 1:100 dilution of 1.34 g/mL CsCl is non-cytotoxic to Sf9 cells. In addition to cytotoxicity, samples that showed no signs of cytotoxicity may show viral infectivity interference by interfering with the ability of the virus to establish infection in the host cells F-TCID50 assays using diluents of 1.34 g/mL CsCl with medium at 1:3.24 (1:100), 1:3.25 (1:320), or 1:3.26 (1:1000) were performed and the result showed that no interference was observed at the dilutions of 1:3.26 and above (Table 8). Therefore, 1:1000 dilution of 1.34 g/mL CsCl was regarded as the non-interfering dilution.

TABLE 8
Interference with rBV infectivity by 1.34 g/cc CsCl

| Dilutions | Log₁₀(TCID₅₀)/mL | LRV |
|---|---|---|
| ESF AF (PC*) | 6.8 ± 0.2 | — |
| 1.34 g/cc CsCl 1:3.2⁴ | 5.5 ± 0.3 | 1.3 ± 0.4 |
| 1.34 g/cc CsCl 1:3.2⁵ | 6.0 ± 0.3 | 0.8 ± 0.4 |
| 1.34 g/cc CsCl 1:3.2⁶ | 6.7 ± 0.2 | 0.1 ± 0.2 |

*PC—positive control

### Toxicity on Sf-9 Cell Cultures and Interference to rBV Infectivity by AVMX Formulation Buffer 1

Similarly, serially diluted AVMX Formulation Buffer 1 with medium was added to cell culture wells and incubated at 28° C. for 6-8 days prior to observation and scoring with a visible/Fluorescent microscope (Amscope, IN300TC-FL). The results showed that no significant morphological changes on the cells were observed at any AVMX Formulation Buffer 1 dilutions and GFP signals were observed in all the wells with added rBV-GFP (data not shown). The interference of rBV infectivity by AVMX Formulation Buffer I was tested at titrations of 1:3.2, 1:3.22, and 1:3.23, and the results showed that no interference was observed at any of the dilutions (Table 9). Therefore, 1:3.2 dilution of AVMX2 formulation buffer was regarded as the non-cytotoxic and non-interfering.

TABLE 9
Inference of rBV infectivity by AVMX formulation buffer 1

| Dilutions | Log₁₀(TCID ₅₀)/mL | LRV |
|---|---|---|
| ESF AF (PC*) | 6.8 ± 0.2 | — |
| AVMX2 buffer 1:3.2 | 6.9 ± 0.2 | 0.0 ± 0.2 |
| AVMX2 buffer 1:3.2² | 6.9 ± 0.1 | 0.0 ± 0.2 |
| AVMX2 buffer 1:3.2³ | 6.7 ± 0.2 | 0.1 ± 0.2 |

*PC—positive control

### Assessment of rBV Clearance by NFR Filtration

After ultracentrifugation, the AAV full band was collected and diluted with AVMX2 formulation buffer to the final AAV titer close to 2.4×10¹² vg/mL. rBV-GFP stock 20-VS-BV-01 was spiked into the processed samples at 2% (v/v) immediately before loading the sample onto an NFR filter. After viral filtration, six fractions were collected and sterilized by 0.22 μm filtration for the F-TCID50 assay as described in Materials and Methods. In this study, AAV in 1.34 g/mL CsCl (1.5 mL) was diluted about 30-fold with AVMX2 formulation buffer (40 mL) before viral filtration. During the F-TCID₅₀ assay, the first column of the plates was prepared containing 1:3.24 (1:100) diluted 1.34 g/cc CsCl or 1:3.2 diluted AVMX Formulation Buffer 1, both were non-cytotoxic to Sf9 cells. However, 1:3.24 and 1:3.25 diluted 1.34 g/mL CsCl were still interfering. Therefore, the limit of detection (LoD) was calculated as ≤2.3 Log₁₀ (TCID50)/mL and the LRV was ≥4.1±0.3 after NFR viral filtration as shown in Table 10. When the process intermediate is less toxic to the assay indicator cells, the assay sensitivity can increase, and the LRV can be larger.

TABLE 10
rBV infectious titers before and after NFR

| Sample | Log₁₀(TCID ₅₀)/mL | LRV |
|---|---|---|
| Before NFR filtration | 6.4 ± 0.3 | — |
| F1 | ≤2.3 | ≥4.1 ± 0.3 |
| F2 | ≤2.3 | ≥4.1 ± 0.3 |
| F3 | ≤2.3 | ≥4.1 ± 0.3 |
| F4 | ≤2.3 | ≥4.1 ± 0.3 |
| F5 | ≤2.3 | ≥4.1 ± 0.3 |
| F6 | ≤2.3 | ≥4.1 ± 0.3 |

Assessment of AAV Recovery and rBV Clearance after NFR Filtration by qPCR

AAV samples were rBV spiked immediately before NFR filtration and six fractions were collected after NFR filtration. The evaluation of AAV recovery and rBV clearance by qPCR is shown in Table 11. More than 80% of AAV was recovered and more than 90% of rBV was removed (LRV of 1). However, the low rBV removal rate as determined by QPCR was partially due to the rBV impurities (rBV-Rep-Cap and rBV-GOI) in the purified AAV samples.

TABLE 11
QPCR analysis of AAV and rBV recovery after NFR filtration

| | Vol | AAV | Total AAV | rBV | Total rBV |
|---|---|---|---|---|---|
| Sample | (mL) | (vg/mL) | (vg) | (vg/mL) | (vg) |
| Before NFR filtration | 40 | 3.92 × 10¹² | 1.57 × 10¹⁴ | 5.63 × 10¹² | 2.25 × 10¹¹ |
| F1 | 5 | 3.06 × 10¹² | 1.53 × 10¹³ | 3.43 × 10¹¹ | 1.72 × 10⁹ |
| F2 | 8 | 4.55 × 10¹² | 3.64 × 10¹³ | 6.92 × 10¹¹ | 5.54 × 10⁹ |
| F3 | 8 | 3.14 × 10¹² | 2.51 × 10¹³ | 4.63 × 10¹¹ | 3.70 × 10⁹ |
| F4 | 7.5 | 3.21 × 10¹² | 2.41 × 10¹³ | 5.10 × 10¹¹ | 3.83 × 10⁹ |
| F5 | 7.5 | 2.54 × 10¹² | 1.91 × 10¹³ | 4.38 × 10¹¹ | 3.28 × 10⁹ |
| F6 | 4 | 1.59 × 10¹² | 6.35 × 10¹² | 1.00 × 10¹¹ | 4.01 × 10⁸ |
| After NFR (Total) | 40 | | 1.26 × 10¹⁴ | | 1.85 × 10¹⁰ |
| Recovery (%) | | | 80.63% | | 8.19% |

As shown in Example 3, 1.34 g/mL CsCl was non-cytotoxic at 1:3.24 (1:100) dilution and above, and non-interfering at 1:3.26 (1:1000) dilution and above. AVMX2 Formulation Buffer 1 was non-cytotoxic and non-interfering at 1:3.2 dilution and above. The results were informative for the research purpose. More representative AAV production samples will be used for rBV clearance by NFR viral filtration step during future GLP and GMP studies. Mock AAV full band will be used for toxicity and interference analyses. The LRV of rBV clearance was ≥4.1±0.3 by NFR filtration. Higher LRV could be obtained if buffer exchange had been performed to remove CsCl toxicity and interfering to increase assay sensitivity. Based on the qPCR results, the AAV recovery after NFR filtration was 80.63%, but the rBV removal was only 91.8%, indicating a LRV of about 1. The inconsistent results between the F-TCID50 and qPCR assays could partially be due to rBV DNA impurities in the partially purified AAV sample.

### Example 4. Viral Inactivation by Low pH Treatment

The BAC-to-AAV technology has been utilized to produce recombinant AAV adeno-associated vector (rAAV). Baculovirus is used for rAAV package during the upstream process in Sf-9 cell culture system. This is the initial source of virus existence as the starting material of manufacturing of rAAV gene therapy product. The baculovirus and Sf-rhabdovirus are enveloped viruses. In order to eliminate any potential viral safety risk associated with the use of rAAV product, a low pH virus inactivation process has been implemented. The current report is the summary of an evaluation study of baculovirus inactivation capacity by the low pH treatment step.

Virus infectivity titer is usually expressed as the median (50%) tissue culture infectious dose (TCID₅₀) when the virus causes distinguishable cytopathic effect (CPE) in the indicator cell culture. In Sf-9 cell culture, the cells are attached on the surface of culture plates as round shapes. However, unlike visible CPE as seen in the cell-based virus assays for detection of mammalian viruses by the TCID₅₀ assay, baculovirus induced morphological changes in Sf-9 cells, such as the increase of cell size and the decrease in viability, are difficult to observe. Therefore, baculovirus assay endpoint may be difficult to determine accurately. To overcome this barrier, Sf-9 cells were inoculated with recombinant baculoviruses (rBV) carrying a green fluorescent protein (GFP) gene. After incubation in Sf-9 cells at 28° C., GFP is expressed and green foci of infected cells can be easily visualized under a fluorescent microscope.

In this study, the F-TCID₅₀ assay was developed and applied to evaluate the baculovirus inactivation during rAAV purification. Firstly, an rBV stock containing a GFP gene was generated and concentrated to a high titer. Secondly, the cell toxicity of the AAVx elution buffer at pH=3.0 was examined to determine the limit of detection of the assay. Finally, the rBV stock was spiked into AAVx eluate, an in-process sample during AAV purification, with pH adjusted to 3.0. The infectivity of the spiked virus was measured before and after the spike using the F-TCID₅₀ assay. The difference of F-TCID₅₀ values between the treated (at selected time points) and non-treated was the baculovirus inactivation expressed in log₁₀ reduction value (LRV). Table 12 illustrates the materials and methods for Example 4.

TABLE 12
Materials and methods

| Product name | Vendor | Cat No. |
|---|---|---|
| Sf-9 | Expression Systems | 94-006F |
| rBV-V445 (GFP) | In-house | 20-VS-BV-01 |
| ESF AF | Expression Systems | 99-300-01 |
| 96-well plate | Fisher Scientific | 07-201-94 |
| Multi-Channel pipettor | Eppendorf | 1200 |
| BSC | Labconco | 362090437 |
| 28° C. incubator | Forma Scientific | 3326 |
| Glycine | Avantor Performance | 4059-02 |
| HCl | Ward's Science | 7647-01-0 |
| microscope | Amscope | IN300TC-FL |
| Countless cell counting | Invitrogen | C10283 |
| chamber slides | | |
| Countess II FL | Life technologies | A27974 |
| −80° C. freezer | Thermo Electron Corp. | 8606 |
| 3 L autoclavable bioreactor | Applikon | ADI |
| | Biotechnology | Z5100002M0 |
| Amico Ultra-15 centrifugal | Merck Millipore | UFC910024 |
| filters | | |

### Toxicity of AAVx Eluate at pH 3.0 on Sf-9 Cells

Sf-9 cells were seeded in 96-well plates at 5×10⁵ cells/mL and 50 μL/well, and incubated at 28° C. for more than 30 min for cells to settle. Serially diluted with EF AF medium at 1:3.2 from 1:3.2 to 1:3.2″, AAVx eluate with pH adjusted to 3.0 was added to the cell culture plates at 50 μL/well with the same dilution on each column. ESF AF medium was added to the last column at 50 μL/well as the negative control. Cells were examined under a visible/Fluorescent microscope after incubation at 28° C. for 6-8 days.

### Recombinant Baculovirus Inactivation by AAVx Eluate pH 3.0

Baculovirus assay indicator cells, Sf-9 cells, were seeded in 96-well plates. rBV-GFP stock was spiked at 1:20 (v/v) in ESF AF medium at the positive control. An aliquot of the virus-spiked medium was immediately quenched with medium at 1:100. Similarly, rBV-GFP stock was spiked in AAVx eluate with pH adjusted to 3.0 at 1:20 (v/v). Aliquots of virus-spiked AAVx eluate pH 3.0 was immediately collected and quenched with medium at 1:100 at selected time points of T=0-(T0), 5-(T5), 10-(T10), 30-(T30), 60-(T60), and 120-min (T120). Finally, an aliquot of the virus-spiked medium was collected and quenched at 1:100 as the End Positive Control (EPC). AAVx eluate without spiked-virus was diluted at 1:100 with medium as the negative control (NC). F-TCID₅₀ assay was performed using 1:3.2 serial dilutions to examine the infectivity of low pH treated rBV-GFP at selected timepoints.

### Toxicity of AAVx Eluate pH 3.0 on Sf-9 Cell Cultures

Baculovirus assay indicator cells, Sf-9 cells, were seeded in 96-well cell culture plates. Serially diluted AAVx column eluate with pH adjusted to 3.0 were added to cell culture wells and incubated at 28° C. for 6-8 days prior to observation and scoring under a fluorescent microscope (Amscope, IN300TC-FL). The results showed significant morphological changes of the Sf-9 cells in the presence of the eluate at pH=3.0 or 3.2 times dilution. However, after 10 times or more dilutions, no morphological changes of the cells were observed compared to the control cells (data not shown). GFP-positive wells were observed in the presence of 10 times of more diluted AAVx eluate at pH=3.0 (data not shown). Infectious titers of baculovirus at the corresponding dilution level would be 1.7 log₁₀ (TCID₅₀)/ml based on Spearman-Karber Equation. Therefore, the limit of detection for the F-TCID₅₀ assay for rBV inactivation by AAVx column eluate pH-3.0 was ≤ 1.7 log₁₀ (TCID₅₀)/mL.

### rBV Inactivation by AAVx Eluate pH 3.0

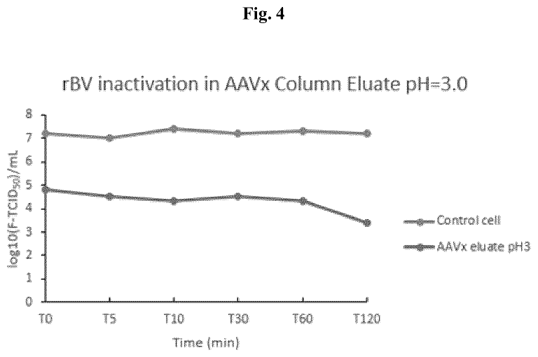

rBV-V445 stock was treated at room temperature with AAVx column eluate pH=3.0 in the virus inactivation kinetic study. With the low pH sample immediately quenched by 1:100 dilution with the medium, significant reduced baculovirus infectivity was detected immediately after the treatment and maintained with the treatment up to 120 min (Table 13 and FIG. 4). In the control culture with untreated baculovirus, the infectivity was not significantly changed (data from a previous assay). The log₁₀ reduction values (LRV) was 2.9±0.4. Therefore, more than 99.7% spiked baculovirus was inactivated by AAVx column eluate pH=3.0.

TABLE 13
Baculovirus inactivation in AAVx column eluate pH = 3.0

| | | AAVx Column Eluate |
|---|---|---|
| Log10(TCID50)/mL | Whole Cell | pH = 3.0 |
| NC | ≤0.7 | ≤1.7 |
| IPC | 7.4 ± 0.2 | 7.4 ± 0.2 |
| EPC | 7.4 ± 0.2 | 7.4 ± 0.2 |
| T0 | 7.2 ± 0.3 | 4.8 ± 0.2 |
| T5 | 7.0 ± 0.2 | 4.5 ± 0.2 |
| T10 | 7.4 ± 0.2 | 4.3 ± 0.2 |
| T30 | 7.2 ± 0.3 | 4.5 ± 0.3 |
| T60 | 7.3 ± 0.2 | 4.3 ± 0.3 |
| T120 | 7.2 ± 0.3 | 3.4 ± 0.2 |
| LRV | 0.2 ± 0.4 | 2.9 ± 0.4 |

The limit of detection of F-TCID₅₀ assay for low pH at 3.0 was ≤1.7 log 10 (TCID₅₀)/mL; The Log reduction value (LRV) for baculovirus inactivation by low pH treatment AAVx eluate pH=3.0 was (2.9±0.4) log₁₀.

Recombinant Baculovirus Containing GFP and Fluorescent TCID₅₀ (F-TCID₅₀) Assay

Recombinant baculovirus expressing GFP (rBV-GFP) was generated using a Bac-to-Bac baculovirus Expression System (Thermo Fischer Scientific, Fremont, CA). Recombinant baculovirus stock was amplified by infecting 50 mL of Sf-9 cell culture at a MOI of 0.1 in a 250-mL Corning bottles at 28° C. with shaking at 190 rpm for three days. The baculovirus was concentrated by ultracentrifugation at 24,000 rpm with a sucrose cushion and the infectious titer of the concentrated rBV-GFP stock was determined by the F-TCID₅₀ assay. Briefly, 50 μL of Sf-9 cells was seeded in 96-well plates at the density of 1.5×10⁴ cells/well and incubated at 28° C. for more than 1 hour for the cells to settle. Make a series of dilutions at 1:10 of the rBV-V445 stock. 50 μL serially diluted virus was added to each well, from low to high, with the same dilution on each column (8 replicates). 50 μL medium was added to the wells in the last column as the negative control. Cells were incubated at 28° C. for 6-8 days prior to assessment for GFP by fluorescent microscopy. A well was counted as positive if one or more green cells were detected, otherwise negative. The infectious titer of the rBV-V445 stock was calculated using Spearman-Karber Equation.

Cell Seeding Density and the Titer of the rBV-GFP Stock by Fluorescent TCID₅₀ Assay

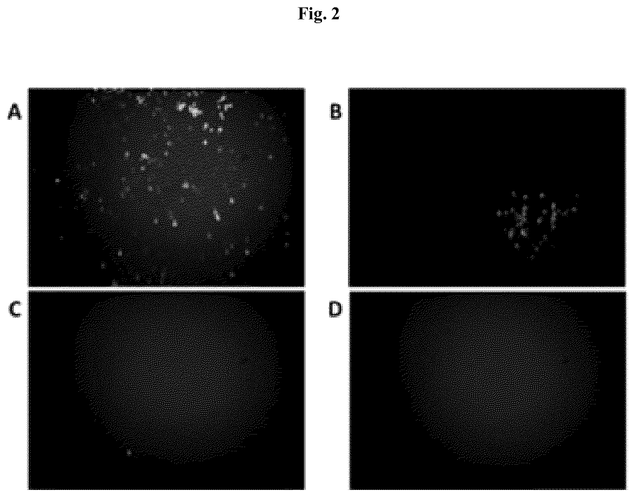

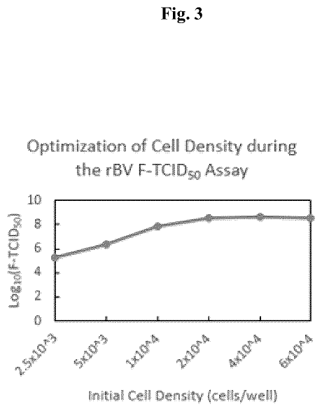

At the end of the assay, wells with green fluorescence foci, which are indicative of infection, were counted as positive, otherwise negative as shown in FIG. 2. To evaluate the optimal cell density at the time of inoculation, Sf-9 cells were seeded in 96-well plates at the concentrations of 2.5×10³, 5×10³, 1×10⁴, 2× 10⁴, 4×10⁴, and 6×10⁴ cells/well. The results showed that the infectious titers obtained at 2× 10⁴, 4× 10⁴, and 6× 10⁴ cells/well were similar and 1-3 log higher than that of the lower cell densities (FIG. 3). Therefore, 2×10⁴ cells/well was selected for the rest of the F-TCID₅₀ assays. The infectious titer of the concentrated rBV-GFP stock was (8.5±0.3) log₁₀(TCID₅₀)/ml at the test condition.

### Example 5. Method of Cell Lysis and Viral Vector Release

This example relates to combination of two methods for lysis of cell wall or membrane in which viral vectors were accumulated after expression and packaging inside host cells. The lysis process was realized with Sarkosyl-related detergents. The other method was the release of viral vectors from cellular particulates, debris, and aggregates using a biodegradable nonionic surfactant. The combination of both lysis of host cells and release of viral vectors was effective and gentle and rendered no impact of the environment.

Gene therapy vectors usually contain components of bacteria, viruses, or other microorganisms and non-biological synthetical vectors. Bacteria supply the plasmids used as small vehicles for trans-genes. Viruses hold considerable appeal as gene therapy vectors, because they are able to incorporate foreign genetic material within the host cell genome. Use of recombinant viral vectors are now the main trends in the gene therapy because the demonstration of safety and efficacy have attained recently. The recombinant viral vectors have the following advantages: demonstrated vectors for gene delivery; easy to scale up to get sufficient product vectors; and a wide range of package capacity from several thousands to ˜40,000 base pairs of double-stranded DNA fragments. In addition, the expression of gene of interest (GOI) lasts very long in vivo up to about 10 years in animal trials.

Though the wide selection of viral vector system, the majority of gene of interests are delivered using adenovirus (AdV) and adeno-associated viral (AAV) vector systems, because these are non-enveloped viral vectors and relatively easy to establish a robust manufacturing process while large quantity of vector doses is required to make. Currently, rAAV gene therapy vectors are popularly manufactured from cell cultures with a human cell line such as HEK293, CHO, A549, or HeLa cells in serum free or serum containing medium in monolayer, suspension of shake flasks, or controlled bioreactors. Helper viruses containing replication and vector packaging of rep/cap necessary elements and GOI are inoculated into the production cultures. After a period of production, the cell culture supernatant is harvested for downstream purification via a series of process steps, such as column chromatography, filtration, or ultra-centrifugation, while the cell paste is discarded. Under this condition, about 80-90% of viral vectors inside cells are discarded with cell paste. The removal of cell paste is usually achieved by the depth filtration or centrifugation.

Another scenario of viral vector purification includes the cell paste collected for viral vector purification, while the cell culture supernatant is discarded. The resultant cell paste is lysed with certain volume or levels of lysis buffer concentrate (20 mM MgCl2, 1% TritonX-100 in 500 mM Tris buffered solution, pH 7.5) and 1% (v/v) of freshly diluted Benzonase® (MilliporeSigam, MA) to digest host-cell DNA, and unpackaged virus DNA are used to the harvested cell culture. After 1 hour of incubation at 37° C. with agitation, concentrated MgSO₄ is added to obtain a final concentration of 37.5 mM. The solution is incubated further for 30 minutes. The addition of MgSO₄ is necessary to avoid AAV aggregation and AAV binding to other cellular components released during lysis.

In the case of large scale of bioprocess, e.g., 100-200,000 L, either purifying viral vector from cell culture supernatant or from cell pastes has the following drawbacks: a dedicated separation is necessary; the separation has to be performed by a continuous centrifugation process, which is a troublesome step in a large scale of biological manufacturing; and loss of viral vectors manufacturing yield. For the case of purification from cell culture supernatant, only a small portion, 10-20% of vectors in the cell culture supernatant and 80-90% of vectors are removed with cell paste. In the case of insect cell culture in particular, the cell pastes harbors about 90% of viral vector.

The current example illustrates purifying viral vector from the whole cell culture without a separation step, via a cell lysis, or depth filtration process. The method for manufacturing and releasing AAV viral particles include: growing Sf9 cells and baculovirus stocks harboring transgene and Rep/Cap; lysing and releasing viral vector by adding lysis buffer to the cell culture at 27±1° C. with rigorous stirring, 300 rpm for 60 min; adding Benzonase at 1-50 IU/mL and MgCl2 to a final concentration of 0.5-50 mM and incubating at 35±2° C. for 30-120 minutes; and removing of cellular particulates and keeping the clarified cell culture fluid (CCF) for further purification. The viral vectors were expected to be more than 70% released in the HCCF which could be used for further purification procedures.

### Adeno-Associated Viral Vector Distribution in Sf9 Cell Culture

Four separate Sf9 cell cultures of 72 hours after infection with baculoviruses harboring with Rep/CAP variants of AVMX-110, AAV2-AMI042, AAV2-AMI-045 and AAV2-AMI046 and gene of interest (GOI) of green fluorescence protein (GFP) were used for analysis of AAV distribution inside cells and supernatant of the cell cultures. The viable cells were counted using hemocytometer method with 0.2% trypan blue. Total cell density and viability of each culture were 4.5×10⁶, 5.6×10⁶, 6.8×10⁶ and 8.3×10⁶ respectively. The determination was performed in duplicate for culture using a 1.0 mL of each Sf9 cell culture. AAV in cells and supernatant were initially separated from each other by centrifugation at 1000 rpm, 5 minute, room temperature (RT) (Beckman Allegra centrifuge, Beckman, Palo Alto, CA). Supernatant were collected separately for AAV titering directly. The pellet of each culture was resuspended and washed once in 1.0 mL of phosphate buffered saline (PBS) (10 phosphate buffer, 150 mM NaCl, pH 7.2, Cat. SH30529.03, GE Healthcare Life Science, Logan, Utah). The resuspension was centrifugated in an Eppendorf centrifuge 5415C at 14,000 rpm for 5 min. Cell pellets were resuspended in 200 μL PBS. AAV titers in the cell culture supernatant and pellets were determined by real time quantitative polymerase chain reaction (qPCR) assay using a primer pair and probe complementary to the region of AAV2 ITR region.

As shown in Table 14, AAV particles were accumulated inside cells, representing 85-95% of total AAV particles, while only 5-15% were detected in cell culture supernatant. The supernatant portion mainly released into cell culture medium due to cell lysis. However, the cellular distribution pattern was not significantly correlated with cell viability, indicating that AAV particles were associated with cellular components or subcellular organelle. Therefore, it was important to lyse cells and release AAV particles into cell culture supernatant prior to purification.

TABLE 14
AAV2 vector distribution in Sf9 cell culture

| Fraction | AVMX-110 | | AMI042 | | AMI045 | | AMI046 | |
|---|---|---|---|---|---|---|---|---|
| AAV serotype | AAV2 | | AAV2 | | AAV2 | | AAV2 | |
| Total cell | 4.5 × 10⁶ | | 5.6 × 10⁶ | | 6.8 × 10⁶ | | 8.3 × 10⁶ | |
| counts | | | | | | | | |
| Viable cells | 1.5 × 10⁶ | | 1.5 × 10⁶ | | 1.8 × 10⁶ | | 3.8 × 10⁶ | |
| Dead cells | 3.0 × 10⁶ | | 3.8 × 10⁶ | | 3.0 × 10⁶ | | 3.9 × 10⁶ | |
| Test | 1 | 2 | 1 | 2 | 1 | 2 | 1 | 2 |
| AAV in | 5.77 × 10¹² | 51.4 × 10¹² | 5.09 × 10¹² | 5.06 × 10¹² | 5.10 × 10¹² | 4.89 × 10¹² | 5.76 × 10¹² | 1.63 × 10¹² |
| pellet | | | | | | | | |
| (vg/mL) | | | | | | | | |
| AAV in | 3.63 × 10¹¹ | 4.87 × 10¹¹ | 2.29 × 10¹¹ | 3.27 × 10¹¹ | 4.53 × 10¹¹ | 1.83 × 10¹² | 5.24 × 10¹¹ | 2.36 × 10¹¹ |
| supernatant | | | | | | | | |
| (vg/mL) | | | | | | | | |
| AAV S/P | 6.29 | 9.47 | 4.50 | 6.46 | 8.88 | 20.4 | 9.09 | 14.47 |
| ratio (%) | | | | | | | | |
| Average | 7.88 | | 5.48 | | 14.66 | | 11.78 | |
| AAV S/P (%) | | | | | | | | |

### Comparison of Sf9 Cell Lysis Using Sodium Lauroyl Sarcosinate (SLS) and Alkyl Polyglucoside (APG) Detergent Solution

Lysis of Sf9 cells was monitored using a turbidity and microscopic methods at visible range of 405 nm wavelength (Table 15). To establish the turbidity assay, a 3.5 mL of Sf9 cell culture was spun down using low speed centrifugation. Pellet was resuspended in 1 mL of PBS containing 1 μL (250 IU) Benzonase and aliquoted into 4 tubes, 190 μL each, for lysis treatment.

TABLE 15
Turbidity measured at absorption at 405 nm (A405)
of Sf9 Cell lysate after SLS-APG treatment

| | | | | | A405 | |
|---|---|---|---|---|---|---|
| | | Cell | Addition of | Final | after lysis | |
| Test | Treatment | suspension | stock solution | volume, | + | − |
| 1 | No detergent | 190 μl | 10 μl PBS | 200 μl | 0.065 | 0.479 |
| 2 | 1% APG | 190 μl | 10 μl 20% | 200 μl | 0.072 | 0.761 |
| | | | APG | | | |
| 3 | 2% APG | 190 μl | 10 μl 40% | 200 μl | 0.059 | 0.724 |
| | | | APG | | | |
| 4 | 1% Triton | 190 μl | 10 μl 20% | 200 μl | 0.086 | 0.49 |
| | X-100 | | Triton X-100 | | | |

From a small volume, the lysis and AAV release process was scaled up for 100-fold using Sf9 cell produced AAV2. In a shake flask, a mixture of 107.2 mL containing 100 mL Sf9 cell culture, 0.20% (w/v) sodium lauryl sarcosinate (SLS), 0.87% APG (w/v), 0.2% SLS (w/v), 5 mM MgCl₂, 25 IU Benzonase, 25 mM Tris pH 7.5, and 37.5 mM NaCl was added for lysing the Sf9 cells. The lysis mixtures were rigorously mixed at 300 rpm for 2 hours prior to filtration with a 0.45 μm Durapore filter (Millipore, MA). The lysate and filtrates were assayed for AAV2 copy number using a real-time quantitative polymerase chain reaction (PCR) with primer pair and probe complementary to the AAV2 ITR sequences.

Lysis of Sf9 Cells with AAV2 Derived Vectors Using 2 L Sf9 AAV2 Culture

The Sf9 cell lysate and AAV release process were performed using a 2 L-scale suspension culture of Sf9-V432AG cells in a 3 L Applikon bioreactor. Briefly, the cell culture was initiated with one liter (L) of Sf9-V432AG cells at 1×10⁶ viable cell (vc)/mL in ESF AF media (Expression Systems, Davis, CA) containing 100 units/mL penicillin and 100 μg/mL streptomycin (Fisher Scientific, CA) in a 3 L Applikon bioreactor with humidity at 100%, temperature at 28° C., stirring speed at 180 rpm, and oxygen level at 21% with a porous sparger. Cell density and viability was monitored daily using Countess II FL cell counter (Life Technologies). Once cell density reached ˜1×10⁷ cells/mL (usually 3 days), 100 mL of Sf9-V432AG cell cultures containing recombinant baculovirus (rBV) carrying a gene of interest (GOI), the Aflibercept (rBV-Aflibercept) and 100 mL of SF9-V432AG cell cultures of recombinant AAV2.N54 (Cap/Rep) (rBV-AAV2.N54) were inoculated into the bioreactor at MOIs of 400-1000. The Sf9-V432AG cell density was at 5×10⁶ cells/mL after addition of 1 L of fresh medium. The inoculated culture mixture, ˜2.2 L were kept for 3 days in the bioreactors with the same parameters except stirring speed at 200 rpm and pH maintained at 6.2. Cell growth was monitored daily. Table 16 illustrates cell lysate and AAV release results.

TABLE 16
Summary of cell lysate and AAV release results

| Production | Lysis | Process | Volume, | AAV titer, | AAV | Recovery |
|---|---|---|---|---|---|---|
| batch | System | intermediate | mL | vg/mL | vg/batch | % |
| Sf9 cells | 1% SLS | Cell culture | 2000 | | | |
| 21-038 | 1% Triton | Lysate | 2590 | 6.18 × 10¹⁰ | 1.95 × 10¹⁴ | 100 |
| | X-100 | DF filtrate | 2540 | 6.25 × 10¹⁰ | 1.57 × 10¹⁴ | 80.5 |
| | | Sterile filtrate | 2510 | 7.54 × 10¹⁰ | 1.57 × 10¹⁴ | 80.4 |
| Sf9 cells | 1% SLS | Cell culture | 2000 | | | |
| 21-045 | 1% Triton | Lysate | 2485 | 6.44 × 10¹¹ | 1.60 × 10¹⁵ | 100 |
| | X-100 | DF filtrate | 2523 | 5.12 × 10¹¹ | 1.29 × 10¹⁵ | 80.6 |
| | | Sterile filtrate | 2510 | 4.76 × 10¹¹ | 1.19 × 10¹⁵ | 74.4 |
| Sf9 cells | 1% SLS | Cell culture | 2000 | | | |
| 21-050 | 1% APG | Lysate | 2520 | 2.43 × 10¹¹ | 6.13 × 10¹⁴ | 100 |
| | | DF filtrate | 2500 | 1.82 × 10¹¹ | 4.56 × 10¹⁴ | 74.4 |
| | | Sterile filtrate | 2480 | 1.95 × 10¹¹ | 4.83 × 10¹⁴ | 78.8 |
| HEK293 | 1% APG | Cell culture | 560 | 8.56 × 10⁹ | 4.8 × 10¹² | |
| 21-051 | | Lysate | 608 | | | |
| | | DF filtrate | N/A | | | |
| | | Sterile filtrate | 608 | | | |

### Comparison Ultracentrifugation and APG SLS Release

One liter of the infected Sf9-V432AG cells were harvested by centrifugation at 3,000 rpm for 10 minutes. Cell pellets were lysed in Sf9 lysis buffer (50 mM Tris-HCl, pH 8.0, 2 mM MgCl2, 1% Sarkosyl, 1% Triton X-100, and 140 units/ml Benzonase). Genomic DNA was digested by incubation at 37° C. for one hour with shaking at 320 rpm. At the end of incubation, sodium chloride was added to adjust the salt concentration of the lysate to about 1 M to further dissociate the AAV vectors from cell matrix. Cell debris was removed by centrifugation at 8,000 rpm for 30 minutes. 50 μL of the clear lysate was aliquoted and saved (20-104 lysate). The rest of the lysate was loaded onto four CsCl step-gradient tubes and subjected to ultracentrifugation at 28,000 rpm for 20 hours in swing bucket rotors. The viral bands were drawn through a syringe with an 18-gauge needle. 50 μL of the combined viral sample from the first spin was aliquoted and saved (20-104 1^{st}). The rest viral sample was loaded onto two second CsCl tubes and subjected to linear-ultracentrifugation at 65,000 rpm for 20 hours. The viral bands were drawn, combined, and diluted to 15 mL with AAV5 Formulation Buffer (20 mM Tris-HCl, pH 8.0, 200 mL NaCl, 1 mM MgCl2, and 0.01% pluronic F-68). The viral solution turned clear after sonicating for 20 s and 50 μL of the viral sample was aliquoted and saved (20-104 2^{nd}). The rest sample was passed through six PD-10 desalting columns (GE HealthCare) to remove the CsCl and detergents and at the same time exchanged to AAV5 Formulation Buffer. A viral sample of 50 μL was aliquoted and saved (20-104 desalting) and the rest was sterilized by passing through a 0.22 μm syringe filter. Quantitative real-time PCR (qPCR) was performed on all the AAV5 samples aliquoted and saved during AAV purification to determine the AAV vector genome copy numbers with ITR primers/probe.

### AAV Vector Production Yield (10 L Scale)

The viral titer and recovery rate of AAV5-AMI082 at each step were determined with ITR primers and probe. Final AAV5-AMI082 vectors were diluted to about 1E+14 vg/mL with AAV5 Formulation Buffer based on the ITR-QPCR titer. Table 17 illustrates yield and recovery of AAV5-AMI082 determined by qPCR.

TABLE 17
Yield and recovery of AAV5-AMI082 determined by qPCR

| | | AAV | Total | Total | | Re- |
|---|---|---|---|---|---|---|
| Sample | | titer | Vol | Yield | Yield | covery |
| I.D. | Name | (vg/mL) | (mL) | (vg) | (vg/L) | (%) |
| 20-104 | 5AMI082 | 6.53E+13 | 100 | 5.63E+15 | 5.63E+15 | 100 |
| Lysate | Lysate | | | | | |
| 20-104 | 5AMI082 | 1.64E+14 | 25 | 4.60E+15 | 4.11E+15 | 73 |
| 1st | 1st | | | | | |

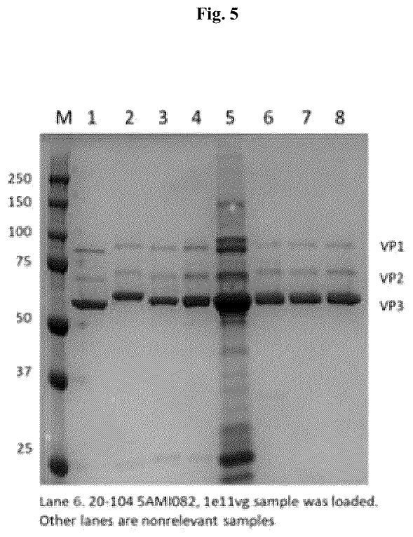

The purified rAAV vectors were separated by the SDS-PAGE following the SOP. The purity of AAV vectors is determined by SimplyBlue Staining assay. Briefly, 26 ul AAV samples (diluted with 1×PBS) were mixed with 10 ul of 4× loading dye plus 4 ul 10× reducing reagent (Invitrogen), and incubate at 95° C. for 5 min. 40 μL final purified AAV5 vectors (about 1e11vg) were then loaded onto a 10% SDS-PAGE gel and run at 100V until the dye reached the bottom of the gel. The gel was stained according to the manufacturer's protocol (Invitrogen). FIG. 5 illustrates the SDS-PAGE and SimplyBlue staining of the purified AAV vectors.

### Example 6. Sf9 Cell Lysis and AAV Release with Non-Ionic Detergent

Described herein is an example for releasing recombinant AAV viral particle (rAAV, also named AMI041, AMI042, AMI045, or AMI046) from a host Sf9 cell. 72 hour cultures of cells transduced with AMI041+31A-V (+V467), AMI042-1A-V (V467), AMI-045, or AMI046 was spun down at 1000 rpm for 5 minutes at room temperature in duplicates. The cells were then contacted with the non-ionic detergent (e.g., APG) for releasing the AAV. qPCR was performed on all samples. Table 18 illustrates yield and recovery of Sf9 cell lysed with the non-ionic detergent.

TABLE 18
illustrates yield and recovery of Sf9 cell lysed with the non-ionic detergent

| Fraction | AMI041 | | AMI042 | | AMI045 | | AMI046 | |
|---|---|---|---|---|---|---|---|---|
| Test | 1 | 2 | 1 | 2 | 1 | 2 | 1 | 1 |
| Pellet (vg/mL) | 5.77 × 10¹² | 5.14 × 10¹² | 5.09 × 10¹² | 5.06 × 10¹² | 5.10 × 10¹² | 4.89 × 10¹² | 5.76 × 10¹² | 1.63 × 10¹² |
| Supernatant(vg/mL) | 3.63 × 10¹¹ | 4.87 × 10¹¹ | 2.29 × 10¹¹ | 3.27 × 10¹¹ | 4.53 × 10¹¹ | 1.83 × 10¹² | 5.24 × 10¹¹ | 2.36 × 10¹¹ |
| S:P ratio (%) | 6.29 | 9.47 | 4.50 | 6.46 | 8.88 | 20.4 | 9.09 | 14.47 |

| | | | | |
|---|---|---|---|---|
| Supernatant | 7.88 | 5.48 | 14.66 | 11.78 |
| Average (%) | | | | |
| Viable cell | 1.5 × 10⁶ | 1.8 × 10⁶ | 3.8 × 10⁶ | 4.4 × 10⁶ |
| Dead cell | 3.0 × 10⁶ | 3.8 × 10⁶ | 3.0 × 10⁶ | 3.9 × 10⁶ |
| Total cell | 4.5 × 10⁶ | 5.6 × 10⁶ | 6.8 × 10⁶ | 8.3 × 10⁶ |
| VC % | 33.33% | 32.1% | 55.88% | 53.01% |

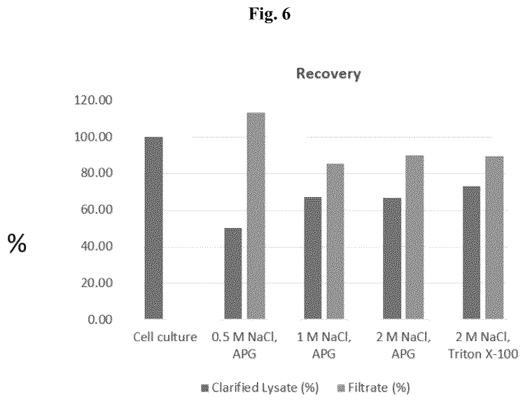

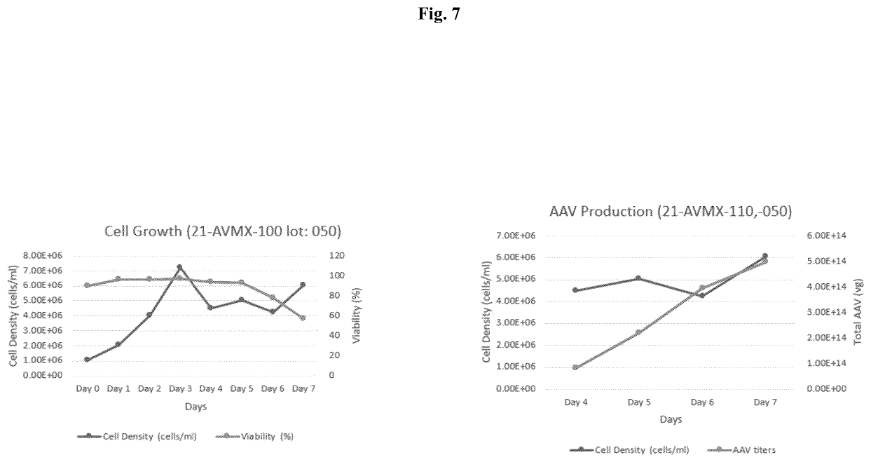

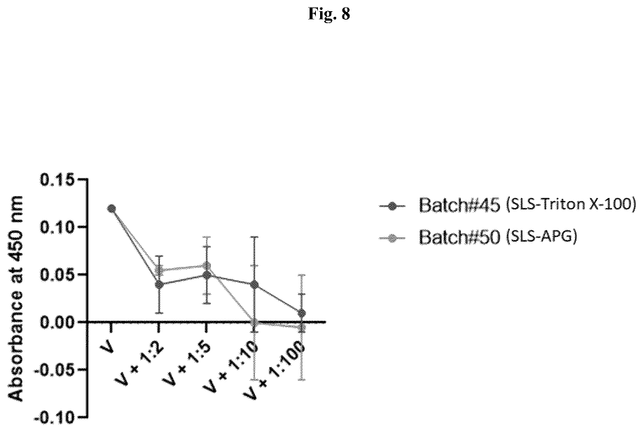

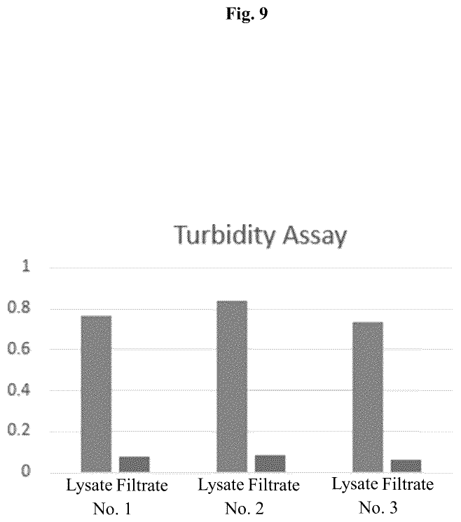

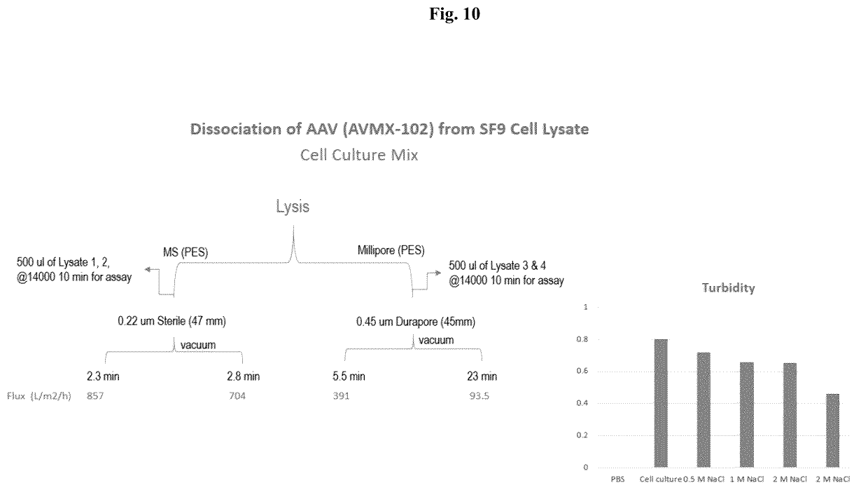

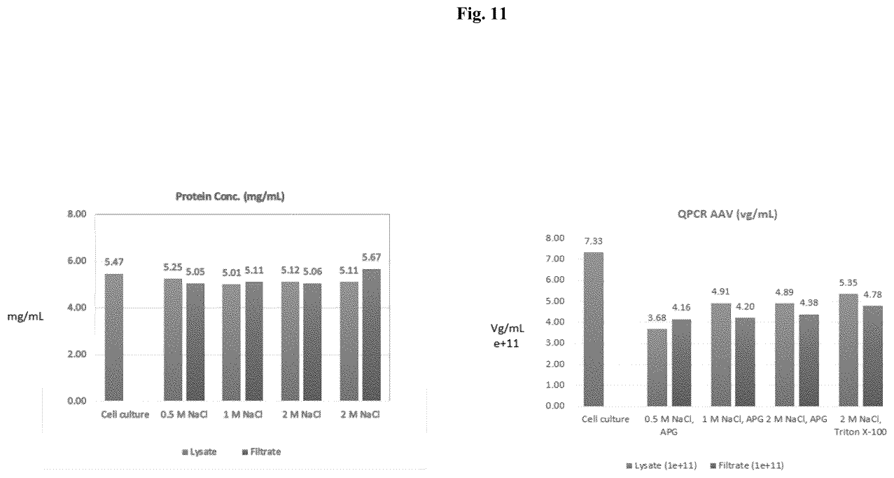

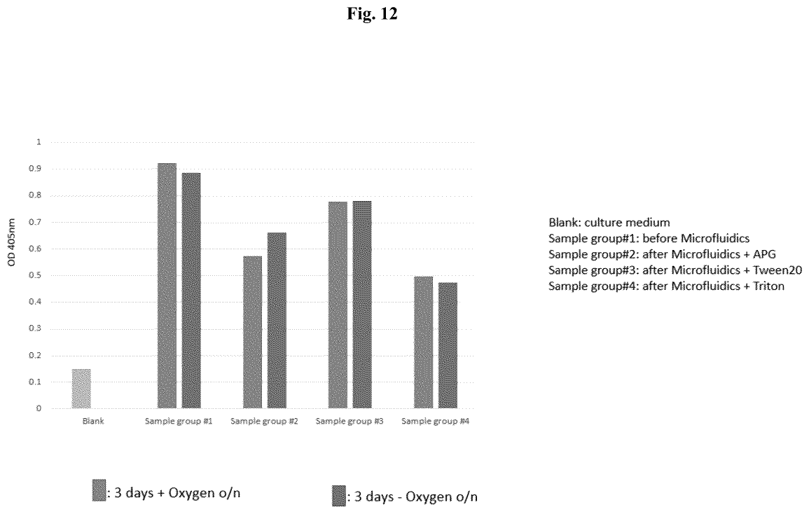

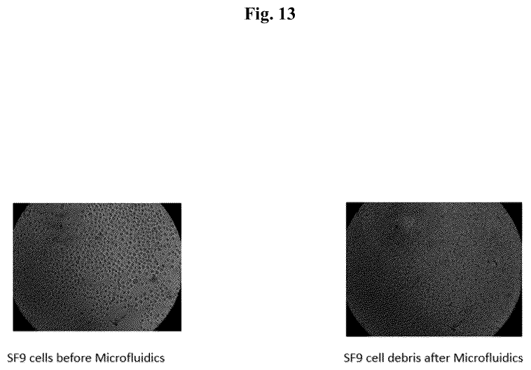

Additionally, the combination of APG (0.87%) and NaCl was examined for yield (Table 19) and recovery (FIG. 6) for releasing the rAAV described herein. Lysate could be clarified with 0.5/0.2 μm pore sized filters. FIG. 7 illustrates Sf cell growth curve (left) and AAV production (right). Table 20 illustrates comparison of AVMX-110 recovery in depth filtration of Sf9 cell culture lysed with sugar-based detergent described herein. Table 21 illustrates expression of Aflibercept by rAAV vectors (AVMX-110) purified from Sf9 cell culture lysed with APG. FIG. 8 illustrates potency of VEGF-Trap expressed by rAAV vectors (AVMX-110) purified from Sf9 cell culture lysed with APG. FIG. 9 illustrates turbidity assay of an exemplary scale-up manufacturing process, where the Sg9 cells were lysed and filtered with 0.45 μm filter (0.45 μm Durapore) yielding a lysate fraction and a filtrate fraction. The two fractions were then subjected to absorbance assay for determining turbidity. Table 22 is numerical representation of FIG. 9. Table 23 illustrates dissociation of AAV (e.g., AVMX-102) from Sf9 cell lysate. FIG. 10 illustrates an exemplary schematic and turbidity graph for dissociation of AAV (AVMX-102) from Sf9 cell lysate. FIG. 11 illustrates yield as determined by protein concentration (left) and qPCR targeting AAV (right). Table 24 illustrates exemplary APG or Trion treatment for lysing Sf9 cells. FIG. 12 illustrates Sf9 cell lysates optical density before and after microfluidics treatment. FIG. 13 illustrates Sf9 cells before microfluidics (left) or Sf9 cell debris after microfluidics (right).

TABLE 19
Recovery % of rAAV released from Sf9 cells during lysis
Recovery % of rAAV Released from Sf9 Cells During Lysis

| | Clarified | | Overall |
|---|---|---|---|
| Sample ID | Lysate (%) | Filtrate (%) | Recovery % |
| Cell culture | 100.00 | | |
| 0.5M NaCl, APG | 50.20 | 113.00 | 56.73 |
| 1M NaCl, APG | 67.00 | 85.40 | 57.22 |
| 2M NaCl, APG | 66.70 | 89.60 | 59.76 |
| 2M NaCl, Triton | 73.00 | 89.50 | 65.34 |
| X-100 | | | |
| Performance | Released | Transmission | Process recovery |
| | | of filter | |

TABLE 20
Comparison of AVMX-110 recovery in depth filtration of Sf9 cell
culture lysed with sugar-based detergent described herein (APG)

| | | | | Total | AAV | Total | |
|---|---|---|---|---|---|---|---|
| | | Detergent | Process | Vol | titer | AAV | Recovery |
| Lot | Lysis Mix | Type | Intermediates | (mL) | (vg/mL) | (vg) | % |
| 21-050 | 1% Sarkosyl | | Lysate | 2520 | 2.43 × 10¹¹ | 6.13 × 10¹⁴ | 100 |
| | (SLS) | | | | | | |
| | 1% APG | Ecofriendly | DF filtrate | 2500 | 1.82 × 10¹¹ | 4.56 × 10¹⁴ | 74.4 |
| | 25 U/mL | | Sterile filtrate | 2480 | 1.95 × 10¹¹ | 4.83 × 10¹⁴ | 78.8 |
| | Benzonase | | | | | | |
| | 5 mM MgCl2 | | | | | | |
| 21-038 | 1% Sarkosyl | | Lysate | 2590 | 6.18E+10 | 1.95E+14 | 100 |
| | 1% Triton | ECHA- | DF filtrate | 2540 | 6.25E+10 | 1.57E+14 | 80.5 |
| | X-100 | REACH | | | | | |
| | 25 U/mL | | Sterile filtrate | 2510 | 7.54E+10 | 1.57E+14 | 80.5 |
| | Benzonase | | | | | | |
| | 5 mM MgCl2 | | | | | | |
| 21-045 | 1% Sarkosyl | | Lysate | 2485 | 6.44E+11 | 1.60E+15 | 100 |
| | 1% Triton | ECHA- | DF filtrate | 2523 | 5.12E+11 | 1.29E+15 | 80.6 |
| | X-100 | REACH | | | | | |
| | 25 U/mL | | Sterile filtrate | 2510 | 4.76E+11 | 1.19E+15 | 74.4 |
| | Benzonase | | | | | | |
| | 5 mM MgCl2 | | | | | | |

TABLE 21
Expression of Aflibercept by rAAV vectors (AVMX-110)
purified from Sf9 cell culture lysed with APG

| | | Aflibercept expression |
|---|---|---|
| Lot | Lysis Detergent | (ug/mL) |
| 21-050 | 1% SLS-APG | 31.6 |
| 21-038 | 1% SLS-Triton X-100 | 31.9 ± 2.1 |
| 21-045 | 1% SLS-Triton X-100 | 33.5 ± 2.6 |

TABLE 22
Turbidity assay of SF9 cell lysate and filtrate

| Treatment | 1 | 2 | 3 |
|---|---|---|---|
| Lysis/Benzonase | no | yes | yes |
| 0.5M NaCl | no | no | yes |
| Absorbance 405 nm | 0.761 | 0.839 | 0.731 |
| Absorbance 450 nm | 0.651 | 0.698 | 0.612 |
| 0.45 μm Durapore | | | |
| Absorbance 405 nm | 0.075 | 0.082 | 0.06 |
| Absorbance 450 nm | 0.039 | 0.04 | 0.04 |

| Treatment | Lysate | Filtrate |
|---|---|---|
| No. 1 | 0.761 | 0.075 |
| No. 2 | 0.839 | 0.082 |
| No. 3 | 0.731 | 0.06 |

TABLE 23
Dissociation of AAV (e.g., AVMX-102) from Sf9 cell lysate

| | Release rAAV from SF9 Cells | | | | |
|---|---|---|---|---|---|
| | APG Conc. % (v/v) or 1.16% (w/v) | | | | |
| | 2% | 2% | 2% | 1% Triton X-100 | |
| | Item Lysate No. | | | | |
| | 1 | 2 | 3 | 4 | Column1 |
| AMI018 (mL) | 50 | 50 | 50 | 50 | |
| 40% APG (mL) | 2.56 | 2.56 | 2.56 | 0 | |
| 20% Triton X-100 (mL) | 0 | 0 | 0 | 2.56 | 1M = 58.5/1000 mL |
| 20% SLS (mL) | 2.56 | 2.56 | 2.56 | 2.56 | 0.5M 56 mL is 1.6 g |
| 500 mM MgCl2 (mL) | 1.1 | 1.1 | 1.1 | 1.1 | 1M 56 mL is 3.2 g |
| NaCl powder (g) | 1.6 | 3.2 | 6.4 | 1.6 | 2M 56 mL is 6.4 g |
| Benzonase (ul) | 10 | 10 | 10 | 10 | |
| 400 rpm 37° C. 90 min | | | | | |
| Filtration, Vacuum | 0.22 um | 0.22 um | 0.45 um | 0.45 um | |
| Filter chemistry | PES | PES | PES | PES | |
| Vendor | MS | | Millipore | | |
| Time to complete (min) | 2.3 | 2.8 | 5.5 | 23 | |
| Filtrate sample ID# | 5 | 6 | 7 | 8 | #9 is cell cult. mix |

TABLE 24
Exemplary APG or Trion treatment for lysing Sf9 cells

| Treatment | 1 | 2 |
|---|---|---|
| | 2% APG | 1% Triton |
| Item | 1% SLS | 1 SLS % |
| AVMX-110, mL | 300 | 300 |
| 40% (v/v) APG, mL | 15.36 | 0 |
| 20% Triton X-100, mL | 0 | 15.39 |
| 20% SLS 9(mL) | 15.39 | 15.39 |
| 5 00 mM MgCl2, mL | 6.6 | 6.6 |
| Benzonase, mL | 0.06 | 0.06 |
| Total | 337.41 | 337.44 |
| 320 rpm 120 min | Y | Y |
| NaCl is added, g | 19.6 | 9.8 |
| 320 rpm 30 min | Y | |
| Millipore OptiScale 0.5/0.2 um | | |

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A method of releasing a plurality of viruses from a host cell, the method comprising:
contacting the host cell with a formulation comprising a sugar-based detergent for lysing the host cell, wherein said contacting with the sugar-based detergent releases at least 50% of the plurality of viruses from the host cell,
wherein the plurality of viruses is encoded by a vector in the host cell, and
wherein the vector encodes at least one therapeutic.

2. The method of claim 1, wherein the plurality of viruses comprises non-enveloped viruses.

3.-4. (canceled)

5. The method of claim 1, wherein the vector encodes at least one virus comprising at least one capsid.

6. The method of claim 5, wherein the at least one capsid comprises a capsid of serotype AAV1, AAV2, AAV3, AAV4, AAV5, AAV6, AAV7, AAV8, AAV9, AAV10, AAV11, AAV12, AAV13, or a combination thereof.

7. (canceled)

8. The method of claim 1, wherein the contacting of the host cell with the formulation inactivates viruses that are different from the plurality of viruses.

9. The method of claim 8, wherein the viruses being inactivated are not encoded by the vector.

10. The method of claim 1, wherein the sugar-based detergent comprises at least one sugar moiety and at least one hydrophobic moiety.

11. (canceled)

12. The method of claim 10, wherein the at least one sugar moiety comprises a sugar polymer.

13. The method of claim 12, wherein the sugar polymer comprises at least two, at least three, at least four, at least five, at least six, at least seven, at least eight, at least nine, at least 10, at least 11, at least 12, or more sugar molecules.

14. The method of claim 10, wherein the at least one sugar moiety comprises at least one glycoside.

15. The method of claim 14, wherein the at least one glycoside is covalently connected to the at least one hydrophobic moiety.

16.-18. (canceled)

19. The method of claim 1, wherein the host cell is a bacteria cell.

20. The method of claim 1, wherein the host cell is an eukaryotic cell.

21. The method of claim 20, wherein the eukaryotic cell is a mammalian cell.

22. The method of claim 20, wherein the eukaryotic cell is a yeast cell.

23. The method of claim 20, wherein the eukaryotic cell is an insect cell.

24. The method of claim 1, wherein the formulation comprises at least one additional active ingredient.

25. The method of claim 24, wherein the at least one additional active ingredient comprises a non-ionic surfactant.

26. The method of claim 24, wherein the at least one additional active ingredient comprises an anionic surfactant.

27. (canceled)

28. A method of releasing a plurality of viruses from a host cell, the method comprising:
contacting the host cell with a sugar-based detergent for lysing the host cell, wherein said contacting with the a sugar-based detergent releases the plurality of viruses that are at least 50% more infective compared to an infectivity of a plurality of comparable viruses that are obtained by releasing the plurality of comparable viruses by a method that does not use the sugar-based detergent, wherein the plurality of viruses are encoded by a vector in the host cell.

29.-40. (canceled)


SEQUENCE LISTING (126 sequences)

SEQ ID NO: 1-41 - skipped (41 entries)

SEQ ID NO: 42 - protein, 8 aa, synthetic construct
  VARIANT 5: note = E, S, or Q
  VARIANT 6: note = A, N, Q, S, T, E, D, K, R, or H
  VARIANT 7: note = A, N, Q, S, T, E, D, K, R, or H
  VARIANT 8: note = K, R, E, or A
LALGXXXX

SEQ ID NO: 43 - protein, 8 aa, synthetic construct
  VARIANT 5: note = E, S, or Q
  VARIANT 6: note = A, N, Q, S, T, E, D, K, R, or H
  VARIANT 7: note = A, N, Q, S, T, E, D, K, R, or H
  VARIANT 8: note = K, R, E, or A
LKLGXXXX

SEQ ID NO: 44 - protein, 8 aa, synthetic construct
  VARIANT 5: note = E, S, or Q
  VARIANT 6: note = A, N, Q, S, T, E, D, K, R, or H
  VARIANT 7: note = A, N, Q, S, T, E, D, K, R, or H
  VARIANT 8: note = K, R, E, or A
VKLGXXXX

SEQ ID NO: 45 - protein, 8 aa, synthetic construct
  VARIANT 5: note = E, S, or Q
  VARIANT 6: note = A, N, Q, S, T, E, D, K, R, or H
  VARIANT 7: note = A, N, Q, S, T, E, D, K, R, or H
  VARIANT 8: note = K, R, E, or A
VKLGXXXX

SEQ ID NO: 46 - protein, 8 aa, synthetic construct
  VARIANT 5: note = E, S, or Q
  VARIANT 6: note = A, N, Q, S, T, E, D, K, R, or H
  VARIANT 8: note = K, R, E, or A
VKLGXXTX

SEQ ID NO: 47 - protein, 8 aa, synthetic construct
  VARIANT 5: note = E, S, or Q
  VARIANT 6: note = A, N, Q, S, T, E, D, K, R, or H
  VARIANT 7: note = A, N, Q, S, T, E, D, K, R, or H
VKLGXXXK

SEQ ID NO: 48 - protein, 8 aa, synthetic construct
  VARIANT 5: note = E, S, or Q
  VARIANT 6: note = A, N, Q, S, T, E, D, K, R, or H
  VARIANT 7: note = A, N, Q, S, T, E, D, K, R, or H
  VARIANT 8: note = K, R, E, or A
LALGXXXX

SEQ ID NO: 49 - protein, 8 aa, synthetic construct
  VARIANT 5: note = E, S, or Q
  VARIANT 6: note = A, N, Q, S, T, E, D, K, R, or H
  VARIANT 8: note = K, R, E, or A
LALGXXTX

SEQ ID NO: 50 - protein, 8 aa, synthetic construct
  VARIANT 5: note = E, S, or Q
  VARIANT 6: note = A, N, Q, S, T, E, D, K, R, or H
  VARIANT 8: note = K, R, E, or A
LALGXXSX

SEQ ID NO: 51 - protein, 8 aa, synthetic construct
  VARIANT 5: note = E, S, or Q
  VARIANT 6: note = A, N, Q, S, T, E, D, K, R, or H
LALGXXTR

SEQ ID NO: 52 - protein, 8 aa, synthetic construct
  VARIANT 5: note = E, S, or Q
  VARIANT 6: note = A, N, Q, S, T, E, D, K, R, or H
LALGXXTK

SEQ ID NO: 53 - protein, 8 aa, synthetic construct
  VARIANT 5: note = E, S, or Q
  VARIANT 6: note = A, N, Q, S, T, E, D, K, R, or H
LALGXXTE

SEQ ID NO: 54 - protein, 8 aa, synthetic construct
  VARIANT 5: note = E, S, or Q
  VARIANT 6: note = A, N, Q, S, T, E, D, K, R, or H
LALGXXTA

SEQ ID NO: 55 - skipped

SEQ ID NO: 56 - protein, 8 aa, synthetic construct
  VARIANT 5: note = E, S, or Q
  VARIANT 6: note = A, N, Q, S, T, E, D, K, R, or H
LALGXXSK

SEQ ID NO: 57 - protein, 8 aa, synthetic construct
  VARIANT 5: note = E, S, or Q
  VARIANT 6: note = A, N, Q, S, T, E, D, K, R, or H
  VARIANT 7: note = A, N, Q, S, T, E, D, K, R, or H
  VARIANT 8: note = K, R, E, or A
LKLGXXXX

SEQ ID NO: 58 - protein, 8 aa, synthetic construct
  VARIANT 5: note = E, S, or Q
  VARIANT 6: note = A, N, Q, S, T, E, D, K, R, or H
  VARIANT 8: note = K, R, E, or A
LKLGXXTX

SEQ ID NO: 59 - protein, 8 aa, synthetic construct
  VARIANT 5: note = E, S, or Q
  VARIANT 6: note = A, N, Q, S, T, E, D, K, R, or H
LKLGXXTK

SEQ ID NO: 60 - skipped

SEQ ID NO: 61 - protein, 10 aa, synthetic construct
LALGETTRPA

SEQ ID NO: 62 - protein, 10 aa, synthetic construct
LALGETTKPA

SEQ ID NO: 63 - protein, 8 aa, synthetic construct
ALGETTKP

SEQ ID NO: 64 - protein, 8 aa, synthetic construct
ALGETTKP

SEQ ID NO: 65 - protein, 10 aa, synthetic construct
LALGETTKPA

SEQ ID NO: 66 - protein, 10 aa, synthetic construct
LKLGQTTKPK

SEQ ID NO: 67 - protein, 10 aa, synthetic construct
LALGQTTKPK

SEQ ID NO: 68 - protein, 10 aa, synthetic construct
LKLGQTTKPA

SEQ ID NO: 69 - protein, 10 aa, synthetic construct
LALGQTTKPA

SEQ ID NO: 70 - protein, 10 aa, synthetic construct
LALGQTTKPA

SEQ ID NO: 71 - protein, 10 aa, synthetic construct
LALGQTTEPA

SEQ ID NO: 72 - protein, 10 aa, synthetic construct
LALGQTTKPA

SEQ ID NO: 73 - protein, 10 aa, synthetic construct
LALGQTTKPA

SEQ ID NO: 74 - protein, 10 aa, synthetic construct
VALGQTTKPA

SEQ ID NO: 75 - protein, 10 aa, synthetic construct
LALGESTARG

SEQ ID NO: 76 - protein, 10 aa, synthetic construct
LALGETSKRA

SEQ ID NO: 77 - protein, 10 aa, synthetic construct
LALGQSTKPA

SEQ ID NO: 78 - protein, 10 aa, synthetic construct
LALGQTTKPA

SEQ ID NO: 79 - protein, 10 aa, synthetic construct
LALGQTTKPA

SEQ ID NO: 80 - protein, 10 aa, synthetic construct
LALGQTTKPA

SEQ ID NO: 81 - protein, 20 aa, synthetic construct
LALGQTTKPALALGQTTKPA

SEQ ID NO: 82 - protein, 10 aa, synthetic construct
VKLGQTTKPA

SEQ ID NO: 83-90 - skipped (8 entries)

SEQ ID NO: 91 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGLALGETTRPATTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 92 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 93 - protein, 743 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSALGETTKPGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 94 - protein, 753 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSALGETTKPGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 95 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLKLGQTTKPKRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 96 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPKRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 97 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLKLGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 98 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 99 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGLALGQTTKPATTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 100 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTEPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 101 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRLALGQTTKPAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 102 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRLALGQTTKPAGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 103 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNVALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 104 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLALGESTARGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 105 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLALGETSKRANRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 106 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLALGQSTKPANRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 107 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSLALGQTTKPAGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 108 - protein, 755 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGLALGQTTKPATTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 109 - protein, 755 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPALALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 110 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNVAKGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 111-120 - skipped (10 entries)

SEQ ID NO: 121 - protein, 735 aa, Adeno-associated virus 2 [sequence omitted]

SEQ ID NO: 122 - protein, 734 aa, Adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 123 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 124 - protein, 736 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 125 - protein, 733 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 126 - protein, 742 aa, Adeno-associated virus
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL